SEQUENCE LISTING (13 sequences)

SEQ ID NO: 1 - DNA, 20 bp, Artificial Sequence
  Synthetic Oligonucleotide
cacagcctcacctcttccta

SEQ ID NO: 2 - DNA, 20 bp, Artificial Sequence
  Synthetic Oligonucleotide
taggaagaggtgaggctgtg

SEQ ID NO: 3 - DNA, 20 bp, Artificial Sequence
  Synthetic Oligonucleotide
tctccacttcaaccggagac

SEQ ID NO: 4 - DNA, 20 bp, Artificial Sequence
  Synthetic Oligonucleotide
gtctccggttgaagtggaga

SEQ ID NO: 5 - DNA, 100 bp, Artificial Sequence
  Synthetic Oligonucleotide
tctccacttcaaccggagacactgtgtcgtctccggttgaagtggagataggaagaggtgaggctgtgcaagaactcacagcctcacctcttcctaaaaa

SEQ ID NO: 6 - DNA, 100 bp, Artificial Sequence
  Synthetic Oligonucleotide
tctccacttcaaccggggcgactgtgtccgccccggttgaagtggagataggaagaggtgaggctgtgcaagaactcacagcctcacctcttcctaaaaa

SEQ ID NO: 7 - DNA, 37 bp, Artificial Sequence
  Synthetic Oligonucleotide
ctgtgcaagaactcacagcctcacctcttcctaaaaa

SEQ ID NO: 8 - DNA, 48 bp, Artificial Sequence
  Synthetic Oligonucleotide
gagacactgtgtcgtctccggttgaagtggagataggaagaggtgagg

SEQ ID NO: 9 - DNA, 48 bp, Artificial Sequence
  Synthetic Oligonucleotide
gggcgactgtgtccgccccggttgaagtggagataggaagaggtgagg

SEQ ID NO: 10 - DNA, 15 bp, Artificial Sequence
  Synthetic Oligonucleotide
tctccacttcaaccg

SEQ ID NO: 11 - DNA, 37 bp, Artificial Sequence; identical to SEQ ID NO: 7
  Synthetic Oligonucleotide
  misc_feature (1)..(1): 5′ phosphorylated
  misc_feature (37)..(37): 3′ thiol modification

SEQ ID NO: 12 - DNA, 48 bp, Artificial Sequence; identical to SEQ ID NO: 8
  Synthetic Oligonucleotide
  misc_feature (1)..(1): 5′ phosphorylated
  misc_feature (33)..(34): Desthiobiotin linker

SEQ ID NO: 13 - DNA, 14 bp, Artificial Sequence
  Synthetic Oligonucleotide
  misc_feature (1)..(1): 5′ methylene blue modification
ctccacttcaaccg

(19) United States
(12) Patent Application Publication
(10) Pub. No.: US 2023/0333128 A1
(43) Pub. Date: Oct. 19, 2023

(54) ASSAY METHOD FOR POINT OF CARE QUANTIFICATION OF AN IMMUNOPHILIN-BINDING IMMUNOSUPPRESSANT DRUG

(71) Applicants: INNAMED, INC., Philadelphia, PA (US); AUBURN UNIVERSITY, Auburn, AL (US)

(72) Inventors: Subramaniam SOMASUNDARAM, Auburn, AL (US); Anup SINGH, Media, PA (US); Eshwar INAPURI, Novi, MI (US); Christopher J. EASLEY, Auburn, AL (US); Hunho JO, Philadelphia, PA (US)

(21) Appl. No.: 17/616,338

(22) PCT Filed: Jun. 26, 2020
(86) PCT No.: PCT/US2020/040010

§ 371 Date: Dec. 3, 2021

Related U.S. Application Data
(60) Provisional application No. 62/867,312, filed on Jun. 27, 2019.

(51) Int. Cl. G01N 33/94; C12Q 1/6804; G01N 33/543

(52) U.S. Cl. CPC G01N 33/9493; C12Q 1/6804; G01N 33/5438

(57) ABSTRACT

Methods and apparatuses, including assays, for detection and/or quantification of tacrolimus that utilize nucleic acid-based nanostructures linked to tacrolimus. In particular, described herein are biosensors having a sensing mechanism configured to react to a tacrolimus-specific binding agent, such as an aptamer or antibody that binds tacrolimus. In some variations, these methods and apparatuses may be configured to provide an electrochemical read-out in which the nucleic acid-based nanostructure is operated in conjunction with a tacrolimus-specific binding agent for sample quantification. A biosensor or a set of biosensors as described herein can be used as a standalone measurement system for tacrolimus and/or as part of a multiplexed cartridge for multiple analytes.

### CROSS REFERENCE TO RELATED APPLICATIONS

This patent application is a national phase application under 35 USC 371 of International Patent No. PCT/US2020/040010, filed Jun. 26, 2020, titled “ASSAY METHOD FOR POINT OF CARE QUANTIFICATION OF AN IMMUNOPHILIN-BINDING IMMUNOSUPPRESSANT DRUG,” now International Patent Application Publication No. WO 2020/264444, which claims priority to U.S. Provisional Patent Application No. 62/867,312, filed on Jun. 27, 2019, and herein incorporated by reference in its entirety.

### SEQUENCE LISTING

The instant application contains a Sequence Listing which has been filed electronically in ASCII format and is hereby incorporated by reference in its entirety. Said ASCII copy, created on Sep. 30, 2020, is named 14729-704-US0_ST25.txt and is 3.35 KB in size.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The methods, apparatuses (e.g., devices, systems, etc.) and compositions described herein relate to an assays capable of quantifying immunophilin-binding immunosuppressant drugs such as tacrolimus, a small molecule immunosuppressant drug, in a test sample. In particular, described herein are electrochemical-based biosensors with a sensing mechanism that uses a nucleic acid-based nanostructure for sample quantification.

### BACKGROUND

Immunophilin-binding immunosuppressant drugs such as tacrolimus are utilized in patients receiving an organ transplant or in some cases in those with autoimmune diseases. They can primarily be effective in treatment or prevention of organ or tissue rejection and graft versus host disease. During immunophilin-binding immunosuppressant drug therapy, monitoring the blood concentration levels of the immunosuppressant is an important aspect of clinical care, because insufficient drug levels may lead to graft (organ or tissue) rejection and excessive levels may lead to undesired side effects such as frequent viral or bacterial infection and toxicity. Blood levels of immunosuppressant are therefore frequently measured so drug dosages can be adjusted to maintain the drug level in an appropriate concentration range.

Tacrolimus is prescribed to about 80% of all organ transplant patients in the United States. The currently accepted analytical approach for measuring tacrolimus concentrations in blood samples is to use LCMS/MS, which is not appropriate for point of care (POC) analysis. Specialized bead-based immunoassay systems also exist, but these require complex and expensive instrumentation. Furthermore, monitoring the blood concentration in transplant patients is essential for dosing due to the narrow range of the tacrolimus therapeutic index.

Despite the need for accurate monitoring, it has not before been possible to quantify tacrolimus using direct electrochemistry or other simple methods suitable for point of care settings. Development of an alternative method based on electrochemistry, which is highly amenable to POC use, could significantly impact human health and improve the quality of care and quality of life for transplant patients.

### SUMMARY OF THE DISCLOSURE

Described herein are methods and apparatuses, including assays, for detection and/or quantification of an immunophilin-binding immunosuppressant drug (such as cyclosporine, tacrolimus, sirolimus, and/or everolimus) from a biological sample that utilize a nucleic acid-based nanostructure linked to an immunophilin-binding immunosuppressant drug antigen molecule for sample quantification. In particular, described herein are biosensors including nucleic acid-based nanostructures linked to an immunophilin-binding immunosuppressant drug antigen. The immunophilin-binding immunosuppressant drug antigen may be a molecule of the immunophilin-binding immunosuppressant drug (e.g., cyclosporine, tacrolimus, sirolimus, and/or everolimus), a metabolite of the immunophilin-binding immunosuppressant drug, a fragment of the immunophilin-binding immunosuppressant drug, or a synthetic form of the immunophilin-binding immunosuppressant drug. Any of these biosensors may include a sensing mechanism configured to react to a binding agent specific to the immunophilin-binding immunosuppressant drug, such as an aptamer or antibody that binds the immunophilin-binding immunosuppressant drug. In particular, described herein are methods and apparatuses (including biosensors) having an electrochemical-based read out of a sensing mechanism that use a nucleic acid-based nanostructure responsive to a binding agent specific to an immunophilin-binding immunosuppressant drug, such as an aptamers or antibody, for sample quantification. In general, the biosensors described herein can be used either as standalone measurement systems or as a component of a testing cartridge that measures fluid analyte concentrations in a multiplexed manner.

For example, described herein are biosensors for detecting an immunophilin-binding immunosuppressant drug (such as cyclosporine, tacrolimus, sirolimus, and/or everolimu). A biosensor may include: a polynucleotide molecule comprising: a first hairpin structural motif, and, in some variations a second (or more) hairpin structural motif; an immunophilin-binding immunosuppressant drug antigen molecule conjugated to the polynucleotide; a signal moiety coupled to the polynucleotide, wherein the signal moiety and the molecule of the immunophilin-binding immunosuppressant drug antigen are in an effective proximity to each other so that a tethered diffusion of the signal moiety is altered when a binding agent that specifically binds to the immunophilin-binding immunosuppressant drug binds to the molecule of the immunophilin-binding immunosuppressant drug antigen that is conjugated to the polynucloetide; and a conductive substrate to which the polynucleotide molecule is tethered.

The immunophilin-binding immunosuppressant drug antigen molecule may be conjugated to the polynucleotide between the first hairpin structural motif and the second hairpin structural motif. In some variations, the immunophilin-binding immunosuppressant drug antigen may be bound to a region 3′ of the first hairpin structural motif; in some variations, the immunophilin-binding immunosuppressant drug antigen may be bound to a region 5′ of the first hairpin structural motif. The immunophilin-binding immunosuppressant drug antigen may be bound to a binding region on the polynucleotide molecule or a region that is modified to couple to the immunophilin-binding immunosuppressant drug antigen. In any of these methods and apparatuses (including biosensors), the immunophilin-binding immunosuppressant drug antigen is a molecule of the immunophilin-binding immunosuppressant drug, or a fragment thereof that binds with specificity (e.g., having a K_{d} of less than K_{d}<1×10⁻⁴, 1×10⁻⁵, 1×10⁻⁶, <1×10⁻⁷, <1×10⁻⁸, etc.). For example, the immunophilin-binding immunosuppressant drug antigen may be an entire molecule of the immunophilin-binding immunosuppressant drug (e.g., tacrolimus). In some variations the immunophilin-binding immunosuppressant drug antigen may be a metabolite of the immunophilin-binding immunosuppressant drug, or a synthetic analog of the immunophilin-binding immunosuppressant drug. For example, the immunophilin-binding immunosuppressant drug antigen may be one of: an immunophilin-binding immunosuppressant drug, a synthetic analog of an immunophilin-binding immunosuppressant drug, a fragment of an immunophilin-binding immunosuppressant drug or an immunophilin-binding immunosuppressant drug metabolite.

In particular, the immunophilin-binding immunosuppressant drug may be tacrolimus. For example, a biosensor may be configured as a biosensor for detecting tacrolimus and may include: a polynucleotide molecule comprising: a first hairpin structural motif, and a second hairpin structural motif; a tacrolimus antigen molecule conjugated to the polynucleotide molecule, wherein the tacrolimus antigen is one of: a tacrolimus molecule, a synthetic analog of tacrolimus or a tacrolimus metabolite; a signal moiety coupled to the polynucleotide, wherein the signal moiety and the tacrolimus antigen molecule are in an effective proximity to each other so that a tethered diffusion of the signal moiety is altered when a tacrolimus-specific binding agent binds to the tacrolimus antigen molecule; and a conductive substrate to which the polynucleotide molecule is tethered. The tacrolimus antigen molecule may be conjugated to the polynucleotide molecule between the first hairpin structural motif and the second hairpin structural motif, as mentioned above.

In any of these biosensor, the signal moiety may be a redox molecule (e.g., the signal moiety may be methylene blue).

In general, the conductive substrate may comprise a conductive metal electrode (e.g., gold, platinum, etc.), or a conductive carbon electrode (or electrode coated in a conductive metal and/or carbon).

Any of these biosensors may include a processor electrically coupled to the conductive substrate and configured to detect a change in the tethered diffusion of the signal moiety. The controller may be configured to use one or more electrochemical techniques, such as chronoamperometry, potentiometry, and square wave voltammetry (SWV) to detect, quantify and/or characterize sample. In some variations the biosensor (or a system including the biosensor) may include two or more electrodes (e.g., a working electrode, one or more reference electrodes, and in some variations a counter electrode); the working electrode and/or the reference electrode may include the polynucleotide molecule coupled to the immunophilin-binding immunosuppressant drug antigen (e.g., a molecule of tacrolimus). In some variations the reference electrode includes a disabled version of the polynucleotide molecule (e.g., the same polynucleotide molecule without the immunophilin-binding immunosuppressant drug antigen and/or without the signal moiety).

Any of these biosensors may include a plurality of polynucleotide molecules each including an immunophilin-binding immunosuppressant drug antigen (e.g., a tacrolimus antigen) molecule conjugated to the polynucleotide molecule, such as conjugated between a pair of hairpin structural motifs, as well as a signal moiety conjugated to an end of each polynucleotide. The plurality of polynucleotide molecules may be tethered to the same conductive substrate.

The controller may include hardware, software and/or firmware to detect the change in the tethered diffusion as described herein. In some variations the controller may include (or be coupled with) a driving circuit configured to apply a driving current (or in some cases, voltage) in a pulse, such as but not limited to a square pulse, at a frequency (e.g., between 1 Hz and 200 Hz, between 1 Hz and 100 Hz, between 5 Hz and 100 Hz, between 5 Hz and 90 Hz, between 20 Hz and 90 Hz, between 50 Hz and 100 Hz, etc.), and one or more detection circuits to receive the signal from the electrode(s) of the biosensor. One or more filter circuits may be used to reduce noise. One or more amplification circuits may be used to amplify the signal(s). The signals may be stores, analyzed and/or transmitted. In some variations the controller may include or be coupled to a communications circuit for transmitting and/or receiving data, including transmitting data (raw or processed) by the biosensor. The communications circuit may be configured for wireless communications. The controller may include one or more processors. The apparatus may also include a power source or a power adapter for use with a power source (e.g., battery, wall power, etc.).

Any of the biosensors described herein may include a linker tethering the polynuclotide to the conductive substrate. In some variations the polynucleotide molecule has a sequence that is 50-100% identical to any one of SEQ ID NOs: 7-8.

Also described herein are systems including any of these biosensors. For example, a system may include a plurality of polynucleotide molecules each including at least one (e.g., a pair of) hairpin structural motifs, a tacrolimus antigen molecule conjugated to each polynucleotide molecule, and a signal moiety conjugated to an end of each polynucleotide, wherein the tacrolimus antigen is one of: a tacrolimus molecule, a synthetic analog of tacrolimus or a tacrolimus metabolite; an electrode, wherein the plurality of polynucleotide molecules are tethered to the electrode; and a processor in electrical communication with the electrode, wherein the processor is configured to detect a change in a tethered diffusion of the signal moieties conjugated to the plurality of polynucleotides.

The signal moieties conjugated to the plurality of polynucleotides may be redox molecules (e.g., methylene blue or any other redox molecule).

In general, any of these systems may also include the binding agent specific to the immunophilin-binding immunosuppressant drug (e.g., a tacrolimus-specific binding agent). The binding agent may be a solution (e.g., an aqueous solution) in concentrated form or a pre-dilluted form. For example, the binding agent specific to the immunophilin-binding immunosuppressant drug (e.g., a tacrolimus-specific binding agent) may comprise an antibody specific to the immunophilin-binding immunosuppressant drug (e.g., tacrolimus) or an aptamer specific to the immunophilin-binding immunosuppressant drug (e.g., tacrolimus). As mentioned above, the polynucleotide molecule may have a sequence that is 50-100% identical to any one of SEQ ID NOs: 7-8.

Also described herein are methods of identifying and/or quantifying an immunophilin-binding immunosuppressant drug. For example, described herein are methods of identifying an immunophilin-binding immunosuppressant drug (such as tacrolimus) in a sample, including the steps of: exposing a sample mixture including the sample (e.g., a blood sample) to a biosensor comprising a signal moiety and an immunophilin-binding immunosuppressant drug antigen molecule (e.g., a tacrolimus antigen molecule) coupled to a polynucleotide molecule. The tacrolimus antigen molecule may be one of: a tacrolimus molecule, a synthetic analog of tacrolimus or a tacrolimus metabolite. The sample or the biosensor may be pre-incubated with a binding agent specific to an immunophilin-binding immunosuppressant drug (such as a tacrolimus-specific binding agent) prior to exposing the sample mixture to the biosensor. The method may further include detecting a change in a tethered diffusion of the signal moiety relative to a surface; and outputting the presence and/or level of tacrolimus in the sample based on change in tethered diffusion of the signal moiety

For example, a method of identifying tacrolimus in a sample may include: contacting the sample with a tacrolimus-specific binding agent to form a sample mixture; exposing the sample mixture to a biosensor comprising a signal moiety and a tacrolimus antigen molecule coupled to a polynucleotide molecule, wherein the tacrolimus antigen is one of: a tacrolimus molecule, a synthetic analog of tacrolimus or a tacrolimus metabolite; detecting a change in a tethered diffusion of the signal moiety relative to a surface; and outputting the presence and/or level of tacrolimus in the sample mixture based on change in tethered diffusion of the signal moiety.

Exposing the sample mixture to the biosensor may comprise exposing the sample mixture to the biosensor, wherein the biosensor comprises a plurality of polynucleotide molecules each including an immunophilin-binding immunosuppressant drug antigen molecule (e.g., a tacrolimus antigen molecule) conjugated to each polynucleotide molecule and the signal moiety conjugated to an end of each polynucleotide molecule. Exposing may include adding, combining, and/or mixing, etc. It may be beneficial to use a concentration of the binding agent specific to the immunophilin-binding immunosuppressant drug that is within the range of detection, such as, e.g., between 1 and 200 nM (e.g., between 1 and 100 nM, between 5 and 100 nM, between 5 and 80 nM, etc.). For example, the tacrolimus-specific binding agent in the sample mixture may be at a concentration of 200 nM or less. For example, the binding agent specific to the immunophilin-binding immunosuppressant drug (e.g., the tacrolimus-specific binding agent) in the sample mixture may be at a concentration of between 1 to 100 nM in the sample mixture.

For example, contacting the sample with the tacrolimus-specific binding agent may include adding one or more of an antibody configured to bind tacrolimus or an aptamer configured to bind tacrolimus.

In any of these methods, detecting a change in a tethered diffusion of the signal moiety relative to a surface may comprise detecting a change in an electrochemical output of the biosensor, wherein the biosensor comprises an electrode to which the polynucleotide molecule is tethered. The biosensor may include a plurality of polynucleotide molecules each including tacrolimus conjugated between a pair of hairpin structural motifs and the signal moiety conjugated to an end of each polynucleotide, wherein the plurality of polynucleotide molecules are tethered to an electrode.

For example, a method of identifying an immunophilin-binding immunosuppressant drug in a sample may include: exposing a sample mixture including the sample to a biosensor comprising a signal moiety coupled to a polynucleotide molecule and an immunophilin-binding immunosuppressant drug antigen molecule coupled to the polynucleotide molecule; wherein either the sample or the biosensor is pre-incubated with a binding agent specific to the immunophilin-binding immunosuppressant drug prior to exposing the sample mixture to the biosensor; detecting a change in a tethered diffusion of the signal moiety relative to a surface; and outputting the presence and/or level of the immunophilin-binding immunosuppressant drug in the sample mixture based on change in tethered diffusion of the signal moiety.

Exposing the sample mixture may comprise exposing the sample mixture to a biosensor comprising a polynucleotide molecule including an immunophilin-binding immunosuppressant drug antigen molecule conjugated between a pair of hairpin structural motifs and a signal moiety conjugated to an end of the polynucleotide. The step of contacting the sample with the binding agent specific to the immunophilin-binding immunosuppressant drug may include adding the binding agent to a concentration of 500 nM or less in the sample mixture. Contacting the sample with the binding agent specific to the immunophilin-binding immunosuppressant drug may include adding the binding agent to a concentration of between 1 to 500 nM in the sample mixture. Contacting the sample with the binding agent specific to the immunophilin-binding immunosuppressant drug may include adding one or more of an antibody configured to bind the immunophilin-binding immunosuppressant drug or an aptamer configured to bind the immunophilin-binding immunosuppressant drug.

Any of these methods may include detecting a change in a tethered diffusion of the signal moiety relative to a surface comprises detecting a change in an electrochemical output of the biosensor, wherein the biosensor comprises an electrode to which the polynucleotide molecule is tethered. The biosensor may include a plurality of polynucleotide molecules each including the immunophilin-binding immunosuppressant drug antigen molecule conjugated between a pair of hairpin structural motifs and the signal moiety conjugated to an end of each polynucleotide, wherein the plurality of polynucleotide molecules are tethered to an electrode. The immunophilin-binding immunosuppressant drug may be one of: cyclosporine, tacrolimus, sirolimus, and everolimus. For example, the immunophilin-binding immunosuppressant drug may be tacrolimus.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A illustrated one example of an apparatus (e.g., system) including a DNA nanostructure including tacrolimus conjugated to a polynucleotide, tacrolimus-specific binding agent and a processor to process signals detected from the DNA nanostructure.

FIG. 1B schematically illustrates a tacrolimus quantification strategy. In this example, the method includes a two-step workflow to quantify the tacrolimus in the lower nanomolar range where a tacrolimus sample is pre-incubated with its respective antibody (e.g., 15 nM) at 37° C. before being added to a thin film gold biosensor which was pre-modified with a DNA nanostructure conjugated to tacrolimus resulting in a competition effect where the binding of the antibody is in equilibrium between the surface bound DNA nanostructure tacrolimus and the free floating tacrolimus in the sample. The DNA nanostructure contained an electrochemical signaling molecule that produced lower currents when antibody was bound, ascertaining the tacrolimus concentration in the sample indirectly.

FIG. 1C shows examples of calibration curves at various tacrolimus concentrations as measured at 15 and 60 minutes after incubation on the sensor surface using the setup as illustrated in FIG. 1B.

FIG. 1D shows a comparison of signal change over time between a sample with 50 nM and 0 nM tacrolimus, where the signal suppression rate was higher with no tacrolimus in the sample as expected due to unhindered binding of antibody to tacrolimus conjugated to the surface confined DNA nanostructure.

FIG. 2 shows serum stability of a tacrolimus DNA nanostructure assay. In FIG. 2, 50 nM tacrolimus (+) mixed with 15 nM tacrolimus antibody was spiked into undiluted serum and in buffer and then added to the sensor surface. Results were then compared to controls with only antibody (−). Expected trends for the competitive assay were observed, where the sample with no tacrolimus demonstrated significantly higher signal suppression as expected.

FIGS. 3A-3D show the time dependence of the tacrolimus DNA nanostructure assay. The figures show (respectively), signals measured by the sensor after 15 minutes (FIG. 3A), 30 min (FIG. 3B), 42 min (FIG. 3C) and 1 hour (FIG. 3D) of incubation at various concentrations of tacrolimus. This data demonstrates an achieved limit of detection around 15 nanomolar.

FIG. 4 illustrates the dependence of the tacrolimus DNA nanostructure assay on the binding partner (e.g., tacrolimus-specific binding agent) selected. FIG. 4 shows two different tacrolimus antibodies (AB1 and AB2) being spiked onto the sensor surface to demonstrate that one has a stronger interaction with the DNA nanostructure and to also show that this interaction is dependent on the concentration used, suggesting there are certain concentrations at which the nanostructure is more sensitive to change than others. This data suggests that the tacrolimus assay can quantify a variety of tacrolimus concentrations and achieve a limit of detection lower than 15 nanomolar by using multiple sensors in each assay and using different conditions (antibody, antibody concentration, incubation time) for each sensor to optimize that sensor's response to a specific, narrow concentration range.

FIG. 5 is a graph showing different concentrations of AB1 in the tacrolimus-specific binding agent for an assay as described in FIG. 4.

FIG. 6 illustrates the strategy behind a tacrolimus DNA nanostructure assay. FIG. 6 demonstrates how current (signal) from the DNA nanostructure decreases upon addition of a tacrolimus antibody due to binding of the tacrolimus conjugated to the nanostructure.

FIG. 7 shows one example of the structure of the tacrolimus after conjugation to the DNA nanostructure. Click chemistry was used to attach the tacrolimus to a central oligonucleotide on the DNA nanostructure as shown.

FIG. 8 illustrates examples of tacrolimus metabolites and synthetic analogs of tacrolimus that may be used as described herein; in particular molecules of these tacrolimus metabolites and synthetic analogs may be coupled to the biosensors as described herein.

FIG. 9 illustrates examples of immunophilin-binding immunosuppressant drugs, including cyclosporine A, tacrolimus, everolimus and sirolimus. This family of immunophilin-binding immunosuppressant drug have similar structures, as shown in FIG. 9 and may all be used as described herein.

### DETAILED DESCRIPTION

Described herein are electrochemical biosensors for measuring immunophilin-binding immunosuppressant drug, such as (but not limited to) tacrolimus, in a biological sample (e.g., fluid sample). The DNA-nanostructure, which may be referred to as a nucleic acid-based nanostructure, can include a single DNA molecule to which an immunophilin-binding immunosuppressant drug antigen specific to the immunophilin-binding immunosuppressant drug to be detected (e.g., tacrolimus, or a metabolite and/or synthetic tacrolimus) is conjugated. The DNA-nanostructure may also include a signal moiety (e.g., an electrochemical and/or in some variations a florescent moiety) and/or a tether linking the DNA-nanostructure to a substrate, such as a conductive substrate or support. The DNA-nanostructure may include one or more (e.g., two) hairpin motifs (loops), and stem regions. For example, in some variations, the DNA-nanostructure to which the immunophilin-binding immunosuppressant drug antigen molecule (e.g., a tacrolimus molecule, a metabolite of tacrolimus or a synthetic tacrolimus) is conjugated includes a single continuous DNA molecule comprising: a first hairpin structural motif, a second hairpin structural motif. The first hairpin structural motif and the second hairpin structural motif may be attached to each other via a segment of single stranded DNA. The DNA-nanostructure may also include a recognition moiety wherein the immunophilin-binding immunosuppressant drug antigen molecule (e.g., tacrolimus, a fragment of tacrolimus, a metabolite of tacrolimus or a synthetic tacrolimus, that binds to a tacrolimus-specific binding agent) is coupled to the single continuous DNA molecule. The DNA-nanostructure also includes a signal moiety. The signal moiety may be coupled to the single continuous DNA molecule and/or formed of the single continuous DNA molecule. In some variations the signal moiety and the immunophilin-binding immunosuppressant drug antigen molecule are in effective proximity to each other, so that the tethered diffusion of the signal moiety is altered when a binding agent specific to an immunophilin-binding immunosuppressant drug binds to the immunophilin-binding immunosuppressant drug antigen molecule on the DNA-nanostructure.

In general, in any of the examples described herein, except where the context makes clear otherwise, when the DNA-nanostructure describes that an immunophilin-binding immunosuppressant drug antigen molecule (e.g., a tacrolimus antigen molecule, such as: a tacrolimus molecule, a fragment of tacrolimus, a synthetic analog of tacrolimus or a tacrolimus metabolite) is included as part of the DNA-nanostructure, it may instead include a modified version of tacrolimus (e.g., a small molecule that also binds specifically to the tacrolimus-specific binding agent. The methods and apparatuses (including biosensors) described herein are shown primarily with examples of tacrolimus; it should be understood that tacrolimus is one specific example of the general category of immunophilin-binding immunosuppressant drugs that may be tested with the methods and apparatuses described herein.

As used herein, “antibody” can refer to a glycoprotein containing at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region and a light chain constant region. The VH and VL regions retain the binding specificity to the antigen and can be further subdivided into regions of hypervariability, termed complementarity determining regions (CDR). The CDRs are interspersed with regions that are more conserved, termed framework regions (FR). Each VH and VL is composed of three CDRs and four framework regions, arranged from amino-terminus to carboxy-terminus in the following order: FR1, CDR1, FR2, CDR2, FR3, CDR3, and FR4. The variable regions of the heavy and light chains contain a binding domain that interacts with an antigen. “Antibody” includes single valent, bivalent and multivalent antibodies. “Antibody” also includes antibody fragments, such as Fab fragments. The term “Fab”, as used herein, refers to antibody fragments including fragments which comprise two N-terminal portions of the heavy chain polypeptide joined by at least one disulfide bridge in the hinge region and two complete light chain polypeptides, where each light chain is complexed with one N-terminal portion of a heavy chain. Fab also includes Fab fragments which comprise all or a large portion of a light chain polypeptide (e.g., V_{L}C_{L}) complexed with the N-terminal portion of a heavy chain polypeptide (e.g., V_{H}C_{H1}). Specifically, the antibodies and antibody fragments described herein may bind specifically to the immunophilin-binding immunosuppressant drug, e.g., including but not limited to tacrolimus.

As used herein, “aptamer” can refer to single-stranded DNA or RNA molecules that can bind to the immunophilin-binding immunosuppressant drug (including but not limited to tacrolimus) with high affinity and specificity. Their specificity and characteristics are typically determined by their primary sequence and their tertiary structure.

As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, 7-7 interactions, cation-7 interactions, anion-7 interactions, polar 7-interactions, and hydrophobic effects.

The term “carboxyl” is as defined above for the formula:

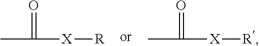

and is defined more specifically by the formula —RivCOOH, wherein Riv is an alkyl, alkenyl, alkynyl, cycloalkyl, heterocyclyl, alkylaryl, arylalkyl, aryl, or heteroaryl. In preferred embodiments, a straight chain or branched chain alkyl, alkenyl, and alkynyl have 30 or fewer carbon atoms in its backbone (e.g., C1-030 for straight chain alkyl, C3-C30 for branched chain alkyl, C2-C30 for straight chain alkenyl and alkynyl, C3-C30 for branched chain alkenyl and alkynyl), preferably 20 or fewer, more preferably 15 or fewer, most preferably 10 or fewer. Likewise, preferred cycloalkyls, heterocyclyls, aryls and heteroaryls have from 3-10 carbon atoms in their ring structure, and more preferably have 5, 6 or 7 carbons in the ring structure. The term “substituted carboxyl” refers to a carboxyl, as defined above, wherein one or more hydrogen atoms in R are substituted. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

As used herein, “deoxyribonucleic acid (DNA)” and “ribonucleic acid (RNA)” can generally refer to any polyribonucleotide or polydeoxribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA) or coding mRNA (messenger RNA).

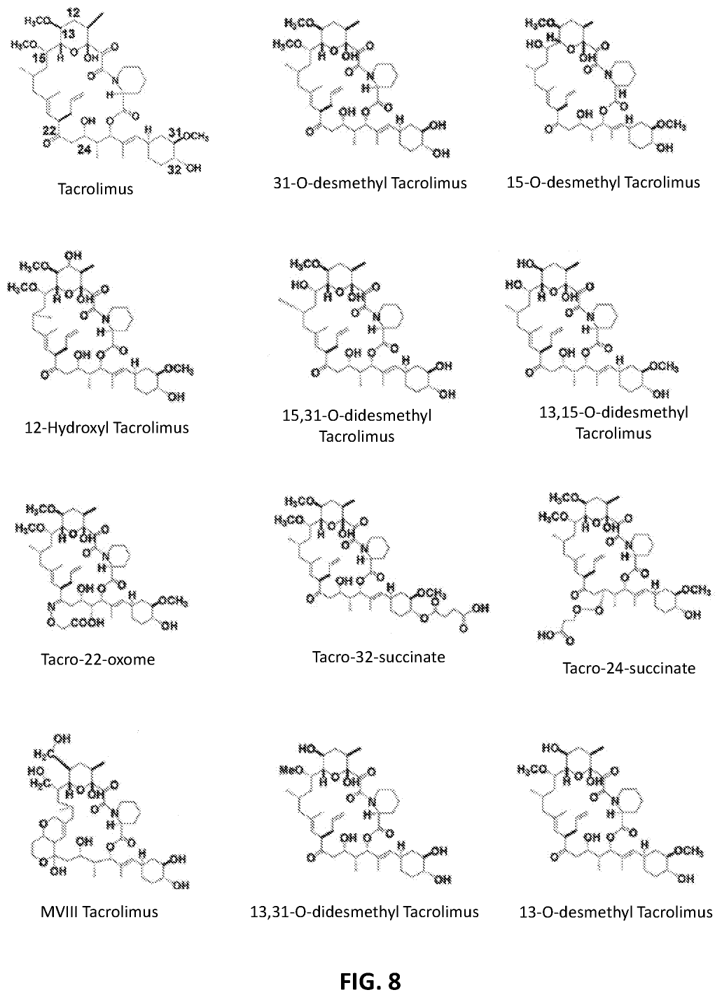

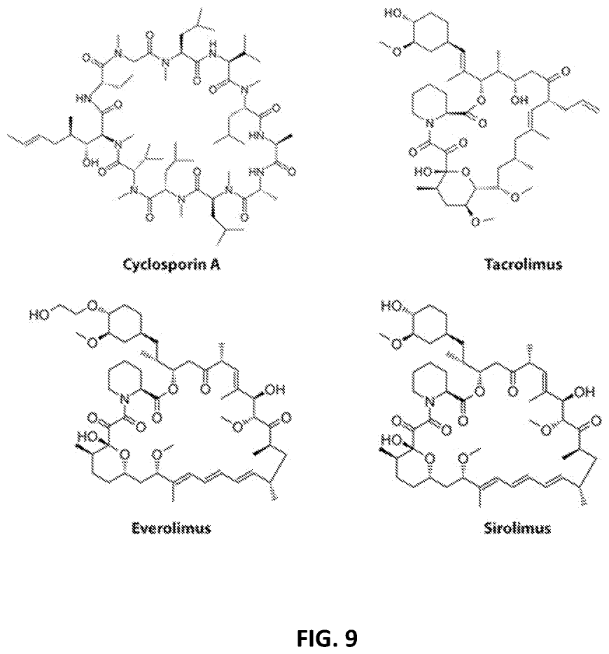

As used herein, “derivative” can refer to any compound having the same or a similar core structure to the compound but having at least one structural difference, including substituting, deleting, and/or adding one or more atoms or functional groups. The term “derivative” does not mean that the derivative is synthesized from the parent compound either as a starting material or intermediate, although this may be the case. The term “derivative” can include prodrugs, or metabolites of the parent compound. Derivatives include compounds in which free amino groups in the parent compound have been derivatized to form amine hydrochlorides, p-toluene sulfoamides, benzoxycarboamides, t-butyloxycarboam ides, thiourethane-type derivatives, trifluoroacetylam ides, chloroacetylam ides, or formam ides. Derivatives may include compounds in which carboxyl groups in the parent compound have been derivatized to form methyl and ethyl esters, or other types of esters or hydrazides. Derivatives include compounds in which hydroxyl groups in the parent compound have been derivatized to form O-acyl or O-alkyl derivatives. Derivatives include compounds in which a hydrogen bond donating group in the parent compound is replaced with another hydrogen bond donating group such as OH, NH, or SH. Derivatives include replacing a hydrogen bond acceptor group in the parent compound with another hydrogen bond acceptor group such as esters, ethers, ketones, carbonates, tertiary amines, imine, thiones, sulfones, tertiary amides, and sulfides. “Derivatives” also includes extensions of the replacement of the cyclopentane ring with saturated or unsaturated cyclohexane or other more complex, e.g., nitrogen-containing rings, and extensions of these rings with side various groups. As used herein a synthetic analog and/or metabolite of tacrolimus may include derivatives of tacrolimus. FIG. 8 illustrates examples of synthetic analogs and metabolites of tacrolimus.

As used herein, “DNA molecule” includes nucleic acids/polynucleotides that are made of DNA.

As used herein, a tacrolimus antigen molecule may refer to a tacrolimus, an antigenic fragment of tacrolimus, a metabolite of tacrolimus, and/or an analog of tacrolimus. In particular, a tacrolimus antigen molecule may refer to a molecule that binds to an antibody specific to tacrolimus (e.g., having an affinity, e.g., K_{d}, of better than 1×10⁻⁶ M) specifically, e.g., with K_{d} of 1×10⁻⁶ M or better (e.g., K_{d}<1×10⁻⁶, <1×10⁻⁷, <1×10⁻⁸, etc.).

In general, the apparatuses (e.g., biosensors), methods and compositions described herein may be used for an immunophilin-binding immunosuppressant drug. Tacrolimus is one example of the general principles (methods and biosensors) described herein. Other immunophilin-binding immunosuppressant drugs may include: cyclosporine, tacrolimus, sirolimus, and everolimus. FIG. 9 illustrates examples of these immunophilin-binding immunosuppressant drugs, and shows their structural similarities. An immunophilin-binding immunosuppressant drug antigen molecule may include any of these immunophilin-binding immunosuppressant drugs, fragments of these that also bind to antibodies against any of these drugs, as well as metabolites of these and synthetic analogs of these. All of the immunophilin-binding immunosuppressant drugs may be used in the same manner as tacrolimus, although the examples of an immunophilin-binding immunosuppressant drug described herein are primarily shown as tacrolimus.

As used herein, “effective proximity” refers to the distance or range of distances that can exists between two or more molecules where an interaction or reaction between the two molecules occurs that generates a measurable response. The effective proximity of a signal moiety and a immunophilin-binding immunosuppressant drug (e.g., tacrolimus) is the distance or range of distances between the signal moieties and the immunophilin-binding immunosuppressant drug where binding of the binding agent specific to the immunophilin-binding immunosuppressant drug by to the immunophilin-binding immunosuppressant drug antigen molecule on the DNA-nanostructure can modulate the tethered diffusion of the signal moiety such that a measurable change in a signal produced, directly or indirectly, by the signal moiety can be detected and/or quantified. In the context of this disclosure, the effective proximity of the signal molecule and a second molecule element (or surface) with which it can chemically react or engage in resonant energy transfer, is the distance or range of distance between the signal molecule and the second molecule or element (e.g., surface) where the interaction can take place such that a measurable change in a signal produced, directly or indirectly, by the signal moiety can be detected and/or quantified.

As used herein, the terms “Fc portion,” “Fc region,” and the like are used interchangeably herein and refer to the fragment crystallizable region of an antibody that interacts with cell surface receptors called Fc receptors and some proteins of the complement system. The IgG Fc region is composed of two identical protein fragments that are derived from the second and third constant domains of the IgG antibody's two heavy chains.

As used herein, “identity,” can refer to a relationship between two or more nucleotide or polypeptide sequences, as determined by comparing the sequences. In the art, “identity” can also refer to the degree of sequence relatedness between polynucleotide or polypeptide sequences as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48: 443-453,) algorithm (e.g., NBLAST, and XBLAST). The default parameters are used to determine the identity for the polypeptides or polynucleotides of the present disclosure, unless stated otherwise.

The term “molecular weight”, as used herein, can generally refer to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, the terms “optional” or “optionally” means that the subsequently described event or circumstance can or cannot occur, and that the description includes instances where said event or circumstance occurs and instances where it does not.

As used herein “peptide” can refer to chains of at least 2 amino acids that are short, relative to a protein or polypeptide.

As used herein, “polymer” refers to a chemical compound formed from a plurality of repeating structural units referred to as monomers “Polymers” are understood to include, but are not limited to, homopolymers, copolymers, such as for example, block, graft, random and alternating copolymers, terpolymers, etc. and blends and modifications thereof. Polymers can be formed by a polymerization reaction in which the plurality of structural units become covalently bonded together. When the monomer units forming the polymer all have the same chemical structure, the polymer is a homopolymer. When the polymer includes two or more monomer units having different chemical structures, the polymer is a copolymer.

As used herein, “polypeptides” or “proteins” refers to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be required for the structure, function, and regulation of the body's cells, tissues, and organs.

As used herein, the term “specific binding” can refer to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, K_{d}, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a K_{d} of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

Specific binding to an immunophilin-binding immunosuppressant drug (or binding specifically to an immunophilin-binding immunosuppressant drug) may refer to the property of the binding agent, such as an antibody, aptamer, etc., to bind to the immunophilin-binding immunosuppressant drug with an affinity (e.g., K_{d}) of 1×10⁻⁶ M or better (e.g., K_{d}<1×10⁻⁶, <1×10⁻⁷, <1×10⁻⁸, etc.). For example, specific binding to tacrolimus (or binding specifically to tacrolimus) may refer to the property of the binding agent, such as an antibody, aptamer, etc., to bind to tacrolimus with an affinity (e.g., K_{d}) of 1×10⁻⁶ M or better (e.g., K_{d}<1×10⁻⁶, <1×10⁻⁷, <1×10⁻⁸, etc.).

As used herein, “surface,” in the context herein, refers to a boundary of a product. The surface can be an interior surface (e.g. the interior boundary of a hollow product), or an exterior or outer boundary or a product. Generally, the surface of a product corresponds to the idealized surface of a three dimensional solid that is topological homeomorphic with the product. The surface can be an exterior surface or an interior surface. An exterior surface forms the outermost layer of a product or device. An interior surface surrounds an inner cavity of a product or device, such as the inner cavity of a tube. As an example, both the outside surface of a tube and the inside surface of a tube are part of the surface of the tube. However, internal surfaces of the product that are not in topological communication with the exterior surface, such as a tube with closed ends, can be excluded as the surface of a product. In some embodiments, an exterior surface of the product is chemically modified, e.g., a surface that can contact a sample component or be coupled to a DNA-nanostructure described herein. In some embodiments, where the product is porous or has holes in its mean (idealized or surface), the internal faces of passages and holes are not considered part of the surface of the product if its opening on the mean surface of the product is less than 1 μm.

As used herein, “substantial” and “substantially,” specify an amount of between 95% and 100%, inclusive, between 96% and 100%, inclusive, between 97% and 100%, inclusive, between 98% to 100%, inclusive, or between 99% to 100%, inclusive.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, “substantially free” can mean an object species is present at non-detectable or trace levels so as not to interfere with the properties of a composition or process.

As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used herein, the term “tethered diffusion” refers to the local diffusion of a moiety that is tethered to a surface, where the moiety is limited from diffusion away from the surface but is diffusing within an approximate hemispherical region of three-dimensional space. In the context of this disclosure, changes in tethered diffusion rates may be observed as changes in electrochemical current measured at a nanostructure-modified electrode when binding agent specific to the immunophilin-binding immunosuppressant drug was either bound or unbound to the immunophilin-binding immunosuppressant drug antigen molecule of the DNA-nanostructure. Various other modes of measurement (optical, vibrational, etc.) could also be used to report tethered diffusion rates.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

The apparatuses for detecting and/or quantifying immunophilin-binding immunosuppressant drugs (e.g., tacrolimus) described herein may be configured as sensors (e.g., electrochemical sensors, florescent sensors) for quantification of biomarkers, which may have a low cost and high adaptability to point-of-care (POC) or point-of-use (POU) setups. These apparatuses may be used for real-time measurements in a variety of sample types, including in the blood of living humans and animals. For example, a DNA-based electrochemical immunophilin-binding immunosuppressant drug (e.g., tacrolimus) sensor may include a DNA-nanostructure that can include a DNA molecule having at least two hairpin structural motifs whose stems are coupled together via a region of un-hybridized DNA and a coupling region to which an immunophilin-binding immunosuppressant drug antigen molecule (e.g., a tacrolimus antigen molecule) may be coupled. The DNA nanostructure may include an anchor recognition region (anchor recognition moiety) that is formed from the DNA molecule, and may be part of the un-hybridized region of DNA that couples the at last two hairpin structural motifs together. The DNA nanostructure may also include a signal moiety that is coupled the DNA molecule forming the DNA nanostructure, such as being coupled to a 3′ terminal end of one of the hairpin motifs (or alternatively a 5′ end). The DNA nanostructure may also include a tether region that is configured to tether the DNA nanostructure to a substrate, and in some variations, to an electrode. For example, the DNA nanostructure may include a tether that may be attach the DNA nanostructure to an electrode.

When the immunophilin-binding immunosuppressant drug antigen molecule coupled to the DNA of the DNA nanostructure is unbound, and not coupled to a binding partner specific to the immunophilin-binding immunosuppressant drug, the DNA-nanostructure may move in 3D space at a first frequency and/or speed defined by its tethered diffusion rate. This movement may be detected in any appropriate manner, such as optically or electrically. For example in variations in which the DNA nanostructure includes a signal moiety that is redox molecule (e.g., methylene blue, nile blue, anthraquinone, ferrocene, ferricyanide/ferrocyanide, etc.), and when the DNA nanostructure is tethered to an electrode surface, the tethered diffusion rate may be detected electrically as the redox molecule moves relative to the electrode. In any of these examples multiple sensor moieties (e.g., multiple redox moieties, such as multiple methylene blues) may be used at the signal moiety.

In any of these variations, the DNA nanostructure may detect binding of the binding partner specific to the immunophilin-binding immunosuppressant drug the tacrolimus coupled to the DNA nanostructure. For example, binding of the tacrolimus-specific binding partner to a tacrolimus antigen molecule on the DNA nanostructure may be measured by detecting and/or measuring a change in the tethered diffusion rate as indicated by the signal moiety. When the tacrolimus antigen molecule coupled to the DNA nanostructure is bound, directly or indirectly, by the tacrolimus-specific binding partner (e.g., antibody, aptamer, etc.), the DNA-nanostructure moves in 3D space at a second frequency and/or speed, defined by its new tethered diffusion rate. The first (unbound) and second (bound) frequencies and/or speeds are different from each other. These frequency/speed can be detected by measuring properties of the signal moiety, either directly and/or indirectly.

Also described herein are methods of making the DNA-nanostructures for detecting an immunophilin-binding immunosuppressant drug and systems including them. These immunophilin-binding immunosuppressant drug-containing DNA-nanostructure (e.g., tacrolimus-containing DNA nanostructure) can be generated by ligating two or more DNA molecules together to form the single molecule DNA-nanostructure. Also described herein are systems that can include a tacrolimus-containing DNA-nanostructure and methods of using these tacrolimus-containing DNA-nanostructures or systems thereof to detect and/or quantify tacrolimus.

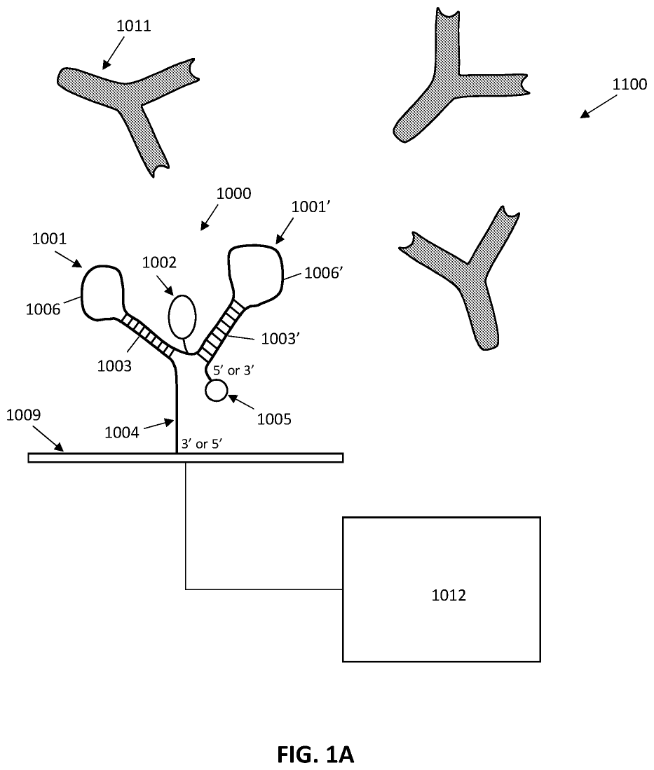

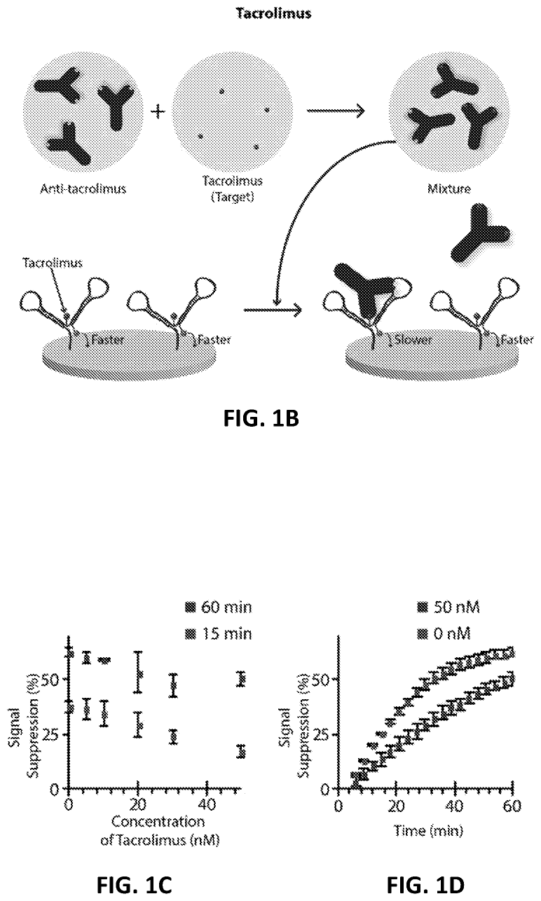

The DNA-nanostructures described herein can detect and quantify an immunophilin-binding immunosuppressant drug when used with a specific binding partner for the immunophilin-binding immunosuppressant drug, such as an antibody or aptamer that binds specifically to the immunophilin-binding immunosuppressant drug. As shown in FIG. 1A and 1B, a DNA-nanostructure 1000 (or a system 1100 including such DNA nanostructures) can be used in assays to detect and/or measure tacrolimus. In FIG. 1A, the DNA-nanostructure 1000 is shown composed of a single continuous DNA molecule that can have specialized structural regions (e.g. hairpin motifs 1001, 1001′, stems 1003, 1003′, an immunophilin-binding immunosuppressant drug coupling region coupled to an immunophilin-binding immunosuppressant drug antigen molecule 1002, a signal moiety 1005, and a tether 1004). The signal moiety 1005 is attached to the rest of the DNA-nanostructure in a position that places the immunophilin-binding immunosuppressant drug antigen molecule 1002 and the signal moiety 1005 in effective proximity to each other such that a change in the binding status of the immunophilin-binding immunosuppressant drug antigen molecule 1002 results in a change in the tethered diffusion of the signal moiety 1005, which can be measured and provide feedback on the binding status. In the system 1100 shown in FIG. 1A, the DNA nanostructure including the immunophilin-binding immunosuppressant drug antigen molecule is tethered to an electrode 1009, and a processor 1012 is coupled to the electrode. The processor may be configured to electrically detect the binding status. The system also includes a binding agent specific to the immunophilin-binding immunosuppressant drug 1011 (shown as an antibody against tacrolimus in this example, where the immunophilin-binding immunosuppressant drug is tacrolimus). Thus, the interaction between the immunophilin-binding immunosuppressant drug and the binding agent specific to the immunophilin-binding immunosuppressant drug may be detected and measured. In this example the tacrolimus is coupled to the polynucleotide to form the DNA-nanostructure 1000; this coupling may be done at any position on the polynucleotide so long as the relative positions between the tacrolimus and the signal moiety 1005 are in effective proximity to each other as described.

The DNA-nanostructure 1000 can include at least two hairpin structural motifs 1001, 1001′ (collectively 1001). The hairpin structural motifs 1001 each can be composed of a stem 1003, 1003′ (collectively 1003) and a loop region 1006, 1006′. The loop 1006, 1006′ in each hairpin motif 1001 contains unpaired nucleotides, and can be, e.g., between 1 to 100 nucleotides (e.g., between 4 to 20 nucleotides), to promote efficient intramolecular hybridization, i.e. hairpin formation, during nanostructure construction or synthesis. The loop in each hairpin motif can be composed of a wide range of nucleotide sequences. The nucleotide sequence can limit or completely eliminate formation of a secondary structure within the loop and also can limit or completely eliminate interactions with any other portion of the nanostructure. A non-limiting example of such a nucleotide sequence can be is a poly-adenosine-monophosphate (polyA) sequence. In some aspects, additional nucleic acid elements can be attached or otherwise incorporated into the loop or other portion of the hairpin motif. In a non-limiting example, the sequence of the first loop 1006 can be 5′- . . . CAA GAA CT . . . -3′ (SEQ ID NO: 1), and one example of the sequence of the loop 1006′ is 5′- . . . ACT GTG TC . . . -3′ (SEQ ID NO: 2). In some aspects, the loop in each hairpin motif can be comprised of a different polymer or biopolymer. One example would be to use a polyethylene glycol (PEG) chain instead of a nucleic acid loop in this region of the nanostructure.

The stem of each hairpin motif is composed of complementary sequence DNA regions that are 90-100% complementary of each other and hybridized through conventional base-pair bonding. The stem of each hairpin motif can contain, e.g., between 2 to 100 nucleotides on each complementary DNA region (e.g., between 10 to 30 nucleotides) to minimize the nanostructure's size while also promoting its assembly and ligation-based synthesis at the surface. In aspects the preferred Tₘ (melting temperature) of a stem and/or hairpin motif can be between 15 to 60 degrees C. prior to ligation and/or hybridization. After ligation and/or hybridization, the complex becomes more stable with a Tₘ typically greater than 70 degrees C. In some aspects, the Tₘ of a of a stem and/or hairpin motif can be about 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, to/or about 60 degrees C. prior to ligation. In some aspects, the complex becomes more stable after ligation and/or hybridization and can have a Tₘ of about 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, or/to about 90 degrees C.

The stem can be composed of a nucleotide sequence. In aspects, the stem nucleotide sequence can promote complementary hybridization (e.g. double-stranded DNA) in the stem region while also limiting or completely eliminating interactions with any other portion of the DNA nanostructure. By way of a non-limiting example, the sequence of the stem can be 5′- . . . CAC AGC CTC ACC TCT TCC TA . . . -3′ (SEQ ID NO: 3) and its complementary sequence 5′- . . . TAG GAA GAG GTG AGG CTG TG . . . -3′; (SEQ ID NO: 4) and the stem can be 5′- . . . TCT CCA CTT CAA CCG GAG AC . . . -3′ (SEQ ID NO: 5) and it's complementary sequence 5′- . . . GTC TCC GGT TGA AGT GGA GA . . . -3′ (SEQ ID NO: 6).

The DNA composing the hairpin motifs can include unmodified or modified nucleotides. Suitable base modifications include, but are not limited to 2-aminopurine, 2,6-diaminopurine, 5-bromo-deoxyuridine, deoxyuridine, inverted dT, inverted Dideoxy-T, Dideoxycytidine, 5-methyl deoxycytidine, deoxyinosine, 5-hydroxybutynl-2′-deoxyuridine, 8-aza-7-deazaguanosine, 5-nitroindole, hydroxymethyl dC, Iso-dC, Iso-dG, 2′ fluoro bases, 2′-O-methoxy-ethyl Bases (2′MOEs). The specific DNA sequence of each hairpin motif can be readily generated by one of ordinary skill in the art based at least upon the parameters and functional aspects of at least the hairpin structural motifs and/or DNA-nanostructure discussed here and elsewhere herein. Commercially available DNA motif and hairpin design tools can be used to generate specific sequences that would form hairpin structures with the appropriate number of paired and unpaired nucleotides according to this disclosure. Such commercially available design tools can include EGNAS (Kick et al., BMC Bioinformatics. 2012. 13:138) or NUPACK (J. N. Zadeh, et al., J Comput Chem, 32:170-173, 2011.). In some aspects, the hairpin motifs have the same DNA sequence. In some aspects the hairpin motifs have a different DNA sequence than each other. In some aspects, the DNA sequence between any two hairpin motifs are completely different from one another. In some aspects, the hairpin motifs differ from each other in 1, 2, 3, 4, 5, 6, 7, 8, 9, 10, 11, 12, 13, 14, 15, 16, 17, 18, 19, 20, 21, 22, 23, 24, 25, or more nucleotides.

The two hairpin motifs can be coupled to each other directly (without unpaired nucleotides) or by an unhybridized (or unpaired) region of DNA (e.g. a region of single stranded DNA) between the stem region of one hairpin motif and the stem region of a second hairpin motif. This coupling region can include 0 to 100 nucleotides, or more nucleotides (e.g., a coupling region may include 0 to 10 nucleotides to minimize the nanostructure size and maintain structural consistency). In aspects, the un-hybridized region can be formed from a 3′ tail of one hairpin motif and a 5′ tail of a second hairpin motif that are ligated together during formation of the DNA-nanostructure. It will be appreciated that where the structure is described with respect to the 5′ and 3′ ends or direction, that the DNA nanostructure may be arranged in the 3′ to 5′ direction in the same manner. As long as the secondary structure can be attained (e.g., hairpin regions separating the tacrolimus and signal moiety so as to translate binding status of the tacrolimus into a different tethered diffusion rate for the signal moiety), the read direction of the underlying nucleotide sequences can be either direction. This is illustrated as such in FIG. 1A, which denotes that the ends of the DNA nanostructure can be either 5′ or 3′ based on the chemical structure of the polynucleotide.

The 3′ (or 5′, when the reverse polynucleotide is considered) end of a stem of one of the DNA structural motifs can be coupled to a tether 1004. The tether 1004 can be a DNA strand that can be configured to couple to a support structure or electrode surface 1009. The length of the tether 1004 can, in aspects where the tether 1004 is coupled to a support or electrode surface 1009, keep the anchor recognition portion at a distance from the support or electrode surface such that the signal moiety is at a suitable distance from the support or electrode surface. The tether can include a linkage from the surface to the double-stranded stem region, such as a polymer chain or carbon chain. The tether can also include unhybridized single stranded DNA. If so, the tether can include 0, 1, 2, 3, 4, 5, 6, 7, 8, 9, 10, 11, 12, 13, 14, 15, 16, 17, 18, 19, or 20 nucleotides. Each of the nucleotides in the tether can be unmodified or modified bases. Suitable base modifications include, but are not limited to 2-aminopurine, 2,6-diaminopurine, 5-bromo-deoxyuridine, deoxyuridine, inverted dT, inverted Dideoxy-T, Dideoxycytidine, 5-methyl deoxycytidine, deoxyinosine, 5-hydroxybutynl-2′-deoxyuridine, 8-aza-7-deazaguanosine, 5-nitroindole, hydroxymethyl dC, Iso-dC, Iso-dG, 2′ fluoro bases, 2′-O-methoxy-ethyl Bases (2′MOEs).

The 3′ (or 5′, when considering the reverse polynucleotide) end of the tether can be optionally modified or coupled to a linking moiety that renders the tether capable of coupling to a support or electrode surface. Suitable linking moieties linking moieties can include, or include after a suitable reaction, a suitable reactive group. Many suitable reactive groups are generally known to those of ordinary skill in the art. Suitable reactive groups include, but are not limited to, a carboxyl group, amino group, aromatic amine group, a chloromethyl group, an amide group, a hydrazide group, a hydroxyl, a thiol, an epoxy or a combination thereof. These groups can be incorporated to the tether. The tether can be attached to a support or electrode surface based, at least in part, on the reactive group used and the chemical nature of the support or electrode surface.

The nucleic acid nanostructure can have any polynucleotide sequence that results in the formation of the general secondary structures as shown in FIG. 1A. For example, a tether can be unattached or can be attached to a surface. The tether is linked to a stem with a loop. A second stem and loop are used to provide structural consistency and to aid in nanostructure assembly and synthesis. The tacrolimus may be positioned near the signal moiety. The positioning of the tacrolimus and signal moiety relative to the nanostructure is not necessarily fixed, although a convenient positioning is to place them in between the two stems as shown in FIG. 1A.

The DNA-nanostructure can have a polynucleotide sequence prior to optional base modification that is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% to 100% identical to any one of SEQ ID NOs: 7-8. It will be appreciated that any primary polynucleotide sequence is acceptable such that it can generate the secondary structure as described elsewhere herein. In some aspects, the DNA-nanostructure can have a polynucleotide sequence prior to any optional base modification that does not share any identity with SEQ ID NOs: 7-8, as long as the primary polynucleotide sequence is acceptable such that it can generate the secondary structure as described elsewhere herein. Suitable primary polynucleotide sequences corresponding to the secondary structures described and demonstrated herein can be designed. A sequence for a tether region may be a linear strand such as polyA, but it is not necessarily limited to this sequence. One example of a sequence that may be used in the examples given herein (SEQ ID NO: 13, SEQ ID NO: 14, and SEQ ID NO: 15). Of course, variations in these sequences are possible.

The systems described herein may include the use of one or more binding agents specific to the immunophilin-binding immunosuppressant drug (e.g., tacrolimus), as described above. Any appropriate binding agent specific to the immunophilin-binding immunosuppressant drug may be used, including, but not limited to antibodies, aptamers, affibodies, proteins, peptides, nucleic acids, polymers, and fragments thereof, that can specifically bind to the immunophilin-binding immunosuppressant drug (e.g., tacrolimus).

Typically, the signal moiety can be capable of producing a signal (e.g. by generating an optical signal) or causing a signal to be produced (e.g. via a redox reaction or resonance-based reaction (e.g. FRET) at an electrode surface or support surface). The tethered diffusion can be measured by the processor 1012 coupled via measuring the signal produced from the signal moiety directly. In variations in which the DNA-nanostructure is not tethered (e.g. is free in a solution), the rate at which the signal moiety moves or its change in position over time can be directly measured by monitoring a signal (e.g. an optical signal) produced from the signal moiety. For example, a fluorescent molecule can be used as the signal moiety, and the fluorescence lifetime of this molecule can be monitored in real time. The fluorescence lifetime will undergo a shift upon binding of a tacrolimus-specific binding agent to the tacrolimus on the DNA nanostructure. Fluorescence lifetime measurements may include time-resolved instrumentation (e.g. with picosecond pulsed lasers and high speed detectors). In another example a fluorescent molecule can be used as the signal moiety, and the fluorescence anisotropy of this molecule can be monitored in real time. The fluorescence anisotropy will undergo a shift upon tacrolimus-specific binding agent binding to the tacrolimus on the DNA nanostrcuture. Fluorescence anisotropy measurements may use polarized optical filters and multiple optical detectors.

A signal produced from the signaling moiety can be directly measured when the signal moiety is attached to a support or an electrode surface. For example, the signal moiety can be configured to produce a signal when it is in proximity to the support or electrode surface such that a resonance-based reaction can occur and result in signal production (e.g. direct fluorescence, total internal reflection fluorescence (TIRF), surface-enhanced fluorescence, etc.) from the signaling moiety. Changes in the frequency or position of interaction of the signaling moiety with the support or electrode surface (e.g. due to the tacrolimus-specific binding agent being bound or not) can result in a change in the output signal from the signaling moiety and can be used to detect and/or quantify presence of the immunophilin-binding immunosuppressant drug, such as tacrolimus.

The tethered diffusion can also be quantified by measuring an output from an electrode, where the electrode is stimulated to produce and/or modulate an output when a signal moiety interacts with an electrode surface. The interaction at the electrode surface can be a chemical (e.g. a redox) reaction that can occur when the signaling moiety is in proximity to the electrode surface such that a reaction can occur. Changes in the frequency or position of interaction of the signaling moiety with the electrode surface (e.g. due to the binding agent specific to the immunophilin-binding immunosuppressant drug being bound or not bound to the immunophilin-binding immunosuppressant drug antigen on the DNA nanostructure) can result in a change in the output signal from the electrode, which can be used to detect and/or quantify presence of immunophilin-binding immunosuppressant drug (e.g., tacrolimus).

In variations in which the electrode is used to detect an electrical signal, e.g., from a signal moiety including a redox molecule, the controller may be configured to operate as (or similar to) a potentiostat, and may control the voltage difference between a working electrode and a reference electrode. The working electrode may include the biosensor electrode(s). These apparatuses may use one or more electrochemical techniques, such as chronoamperometry, potentiometry, and square wave voltammetry (SWV) to detect, quantify and/or characterize sample, e.g., to determine the presence and/or quantity of an immunophilin-binding immunosuppressant drug such as tacrolimus. A square wave voltage (SWV) may be used due to its excellent sensitivity, rejection of background currents and speed. In SWV, the excitation signal consists of a symmetrical square-wave pulse of amplitude E_{sw} superimposed on a staircase waveform of step height ΔE, where the forward pulse of the square wave coincides with the staircase step. The net current, iₙₑₜ, is obtained by taking the difference between the forward and reverse currents (i_{for}−iᵣₑᵥ) and is centered on the redox potential. The peak height is directly proportional to the concentration of the electroactive species (e.g. protein, small molecule, metabolite, etc.).

In one example, an immunophilin-binding immunosuppressant drug such as tacrolimus may be quantified using a DNA nanostructure (e.g., sensor) as described herein, by square-wave voltammetry.

Any of the apparatuses (e.g., systems) described herein may include two or more electrodes, including a working electrode and one or more reference electrodes (and in some variations, particularly those configured to perform voltammetric measurements, may include a counter electrode). In any of these methods and apparatuses, the apparatus may be configured to perform voltammetric measurements by sweeping the potential of the working electrode with respect to the reference and counter electrode. For example, to measure methylene blue—a redox molecule which has a redox potential of −0.22 V vs silver/silver chloride reference—the potential will be typically swept from −0.45 to 0 V and current will be continuously measured. The measured current may be plotted across the applied potential as I vs V plot. In the case of methylene blue measurement, current (I) is typically observed around −0.22 V (typically the average of −0.35 to −0.15 V is analyzed) is from the oxidation of methylene blue.

In any of the methods and apparatuses described herein, square-wave voltammetry may be used. Square-wave voltammetry is a pulse voltammetry technique which is sensitive and capable of measuring the lower concentration of redox molecule (even in nM range). In this method, the potential is swept from initial to final in a square-wave fashion and the measurements are done at the end before applying the pulse. In one square-wave two measurements are done I_{fr} and Iᵣₑ, the difference of these current (I_{diff}) is plotted against the potential as square-wave voltammetry plot. The waiting time between the pulse and the measurement is determined by square-wave voltammetry frequency used. For instance, if 100 Hz is used as square-wave voltammetry frequency the measurement will be done after the pulse with the wait time of 5 ms (two measurements in one cycle).

For example, the following square-wave voltammetry parameters may be used (e.g., for tacrolimus measurements): Initial voltage: −0.45 V, final voltage: 0.00 V, Frequency: 70 Hz, Pulse height: 25 mV, Step size: 2 mV. Any appropriate range of values may be used. For example, the initial voltage may be between −0.1 V and −1 V; the frequency may be between about 10 Hz and 200 Hz (e.g., between about 10 Hz and about 120 Hz, between about 20 Hz and 100 Hz, etc.). The pulse height may be between 1 mV and 60 mV, between 10 mV and 50 mV, between 5 mV and 40 mV, between 15 mV and 35 mV, etc. The step size may be between 0.1 mV and 5 mV (e.g., between 0.5 mV and 4 mV, between 1 mV and 5 mV, between 1 mV and 3 mV, etc.).

Measurement may be done before and after the sample mixture (e.g., sample plus tacrolimus antibody) is incubated on the electrode. A calibration curve may be determined; for the calibration curve, the percentage of peak height change may be plotted vs concentration of immunophilin-binding immunosuppressant drug (e.g., tacrolimus). When using the above-mentioned parameters (specifically 70 Hz) signal suppression is observed on tacrolimus antibody binding.

Suitable signaling moieties include redox molecules and optically active molecules. Suitable redox molecules include, but are not limited to methylene blue, Nile blue, anthraquinone, ferrocene, ferricyanide/ferrocyanide, etc. Suitable optically active molecules include, but are not limited to, any light emitting (fluorescent, infrared, ultra-violet, etc.) small molecule compound include chemical compounds and quantum dots. Fluorescent small molecule compounds are commercially available and generally known in the art and include, but are not limited to, fluorescein, carboxyfluorescein (FAM), rhodamine, carboxy-X-rhodamine (ROX) coumarin, cyanine, Oregon green, eosin, Texas Red, indocarbocyanine, oxacarbocyanine, thiacarbocyanine, merocyanine, squaraine and derivatives thereof, squaraine rotaxane and derivatives thereof, naphthalene, TAMRA, VIC, TET, Cy3, Cy5, TyE 563, Yakima Yellow, HEX, TEX 615, TYE 665, TYE 705, AlexaFlour compounds (e.g. Alexa Flour 488, 532, 546, 594, 647, 660, 750) LI-Cor IRdyes (e.g. 5′ IREDye 700 or 800), ATTO Dyes (e.g. ATTO 488, 532, 550, 565, Rho101, 590, 633, 647), SeTau lifetime and polarization labels (e.g. SeTau-380, 405, 425, 647, 665, 670, 680), and WelIRED dyes (WelIRED D4 Dye, WelIRED D3 Dye, WelIRED D2 Dye). Other suitable optically active molecules will be appreciated by one of ordinary skill in the art in view of this description herein.

As described above, the DNA-nanostructure can be attached to a support structure or an electrode surface. The support structure or electrode can be any suitable shape or design. The electrode may be a support structure. The DNA-nanostructure can be attached to the support structure or an electrode surface via the tether. As previously discussed, the tether can have a 3′ linker or modified nucleotide(s) that can provide a reactive group that can be used to attach the tether to the support structure or an electrode surface.

Suitable support structures can be any solid or semi-solid (e.g. hydrogel) material. Suitable materials include glass, ceramics, metals, metal oxides, metal alloys, polymeric materials (polymers, copolymers, composite polymers, polymer blends, etc.), mixtures thereof and composites thereof. Metals can include, but are not limited to, the alkali metals, alkaline earth metals, transition metals, rare earth metals, combinations thereof, mixtures thereof, and composites thereof. In aspects, the metal or metal composite, oxide, alloy, or mixture thereof can be or include gold, aluminum, copper, iron, lead, silver, platinum, zinc, and/or nickel. Suitable polymeric materials can include, but are not limited to, natural and synthetic polymeric materials. Natural polymeric materials can include, but are not limited to, polysaccharides, natural rubber, polylacticacid, polylysine, polyglutamate, polyornithine, polyarginine, polyaspartate, polyhistidine, polylactide, etc. Synthetic polymers include, but are not limited to, polyethylene, polypropolyene, polystyrene, polyvinyl chloride, synthetic rubber, phenol formaldehyde resin, silicone, polyacrylonitrile, polystyrene, polytetrafluoroethylene, polyurethanes, polyethylene terephthalate, and combinations, copolymers, and blends thereof. The polymeric material can be a thermoplastic, thermoset, elastomer, or a permissible combination thereof.

The electrode and/or electrode surface can be made of any suitable material. In aspects, the suitable material is electrically conductive. In some aspects, electrode and/or electrode surface can be made of a metal, metal oxide, metal composite, metal alloy, or a combination thereof. Metals can include, but are not limited to, the alkali metals, alkaline earth metals, transition metals, rare earth metals, combinations thereof, mixtures thereof, and composites thereof. In aspects, the metal or metal composite, oxide, alloy, or mixture thereof can be or include gold, aluminum, copper, iron, lead, silver, platinum, zinc, and/or nickel.

The DNA-based nanostructures described herein (DNA nanostructures) can be formed of three separate components that can be brought together to form the single molecule DNA-nanostructure. For example, a first partial hairpin DNA (e.g., with about 1-50 bp, such as between 3-15 bases, hybridized in the stem region) can be generated by any suitable DNA synthesis technology (e.g. any recombinant or de novo synthesis technique). The 3′ (or 5′ end when considering the reverse) end can be optionally modified with a suitable linker or contain one or more modified nucleotides, where the linker or modification provides a reactive group to allow for optional attachment to a support structure or electrode surface. If the DNA-nanostructure is optionally coupled to a support or electrode surface, the first partial hairpin DNA can be coupled to the support or electrode surface prior to further assembly of the DNA-nanostructure. The first partial hairpin can have a sequence prior to optional 3′ (or 5′ when considering the reverse) terminal base modification that is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% to 100% identical to SEQ ID NO: 9. In aspects, the first partial hairpin can have a sequence prior to base modification that is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 9. It will be appreciated that any primary polynucleotide sequence is acceptable such that it can be assembled with other DNA-nanostructure components described elsewhere herein and generate the secondary structure. The first partial hairpin can have a polynucleotide sequence prior to any optional base modification that does not share any identity with SEQ ID NO.: 9, as long as the primary polynucleotide sequence is acceptable such that it can be assembled with other DNA-nanostructure components described elsewhere herein and generate the secondary structure as described elsewhere herein.

A second partial hairpin DNA can be generated by any suitable DNA synthesis technology (e.g. any recombinant or de novo synthesis technique). The second partial hairpin DNA can have one or more modified bases that can incorporate a reactive group or a linker that can contain a reactive group that can be capable of coupling tacrolimus. The tacrolimus may be coupled to the modified base. For example, the modified base can be a non-5′ (or 3′ when considering the reverse) terminal base. The modified base can be any internal (i.e., not the terminal 5′ or 3′ base). Thus, the tacrolimus may be incorporated in the DNA-nanostructure once assembled without needing further post-assembly reactions to attach the tacrolimus. Suitable base modifications include, but are not limited to 2-aminopurine, 2,6-diaminopurine, 5-bromo-deoxyuridine, deoxyuridine, inverted dT, inverted Dideoxy-T, Dideoxycytidine, 5-methyl deoxycytidine, deoxyinosine, 5-hydroxybutynl-2′-deoxyuridine, 8-aza-7-deazaguanosine, 5-nitroindole, hydroxymethyl dC, Iso-dC, Iso-dG, 2′ fluoro bases, 2′-O-methoxy-ethyl Bases (2′MOEs). Suitable reactive groups include, but are not limited to, a carboxyl group, amino group, aromatic amine group, a chloromethyl group, an amide group, a hydrazide group, a hydroxyl, a thiol, an epoxy, an azide, click chemistry modifications, or a combination thereof.

The second partial hairpin can have a sequence prior to base modification that is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% to 100% identical to any one of SEQ ID NOs: 10-11. The second partial hairpin can have a sequence prior to base modification that is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to any one of SEQ ID NOs: 10-11. Any primary polynucleotide sequence is acceptable such that it can be assembled with other DNA-nanostructure components described elsewhere herein and generate the secondary structure as described elsewhere herein. The second partial hairpin can have a polynucleotide sequence prior to any optional base modification that does not share any identity with any of SEQ ID NOs.: 10-11, as long as the primary polynucleotide sequence is acceptable such that it can be assembled with other DNA-nanostructure components described elsewhere herein and generate the secondary structure as described elsewhere herein.

A single stranded DNA molecule can be generated that is configured to partially hybridize to the second partial hairpin DNA molecule. The single stranded DNA molecule can be generated by any suitable DNA synthesis technology (e.g. any recombinant or de novo synthesis technique). The single stranded DNA molecule can include a signal molecule. The signal molecule can be coupled to the 5′ end of the single stranded DNA molecule. Suitable signal molecules are described elsewhere herein. The signal molecule can be coupled to the 5′ end via coupling to a modified or unmodified 5′ terminal base or linker attached to 5′ terminal base. The modification can provide a reactive group that can be used to couple, directly or indirectly via a linker, a signal moiety to the 5′ terminal base. In aspects, the linker can contain a reactive group that can couple to a signal moiety. Suitable base modifications include, but are not limited to 2-aminopurine, 2,6-diaminopurine, 5-bromo-deoxyuridine, deoxyuridine, inverted dT, inverted Dideoxy-T, Dideoxycytidine, 5-methyl deoxycytidine, deoxyinosine, 5-hydroxybutynl-2′-deoxyuridine, 8-aza-7-deazaguanosine, 5-nitroindole, hydroxymethyl dC, Iso-dC, Iso-dG, 2′ fluoro bases, 2′-O-methoxy-ethyl Bases (2′MOEs). Many suitable reactive groups are generally known to those of ordinary skill in the art. Suitable reactive groups include, but are not limited to, a carboxyl group, amino group, aromatic amine group, a chloromethyl group, an amide group, a hydrazide group, a hydroxyl, a thiol, an epoxy, an azide, click chemistry modifications, or a combination thereof. The single stranded DNA molecule can be modified prior to DNA-nanostructure assembly to include a signal moiety. The signal moiety can be coupled to the DNA-nanostructure after assembly.

The single stranded DNA molecule can have a sequence prior to optional 3′ (or 5′ when considering the reverse) terminal base modification that is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% to 100% identical to SEQ ID NO: 12. The single stranded DNA molecule can have a sequence prior to base modification that is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical SEQ ID NO: 12. The single stranded DNA molecule can have a polynucleotide sequence prior to any optional base modification that does not share any identity with SEQ ID NO.: 12, as long as the primary polynucleotide sequence is acceptable such that it can be assembled with other DNA-nanostructure components described elsewhere herein and generate the secondary structure as described elsewhere herein.

The assembly of the DNA-nanostructure from the three components (whether coupled to a support structure or electrode surface or not) can occur in some aspects via self-assembly driven by equilibrium. A suitable DNA ligase can be used to assemble the components of the DNA-nanostructure in a non-equilibrium manner. Suitable DNA ligases include, but are not limited to, T4 DNA ligase, T7 DNA ligase, Taq DNA ligase, Electroligase, HiFi Taq DNA ligase, NxGen T4 DNA ligase, Ampligase, and T4 RNA ligase 2. The three components of the DNA-nanostructure can be included in a reaction and contacted with an amount of a suitable DNA ligase and, under suitable reaction conditions, allowed to react with the DNA ligase to form the single molecule DNA-nanostructure. The volume and the concentration can be maneuvered, e.g., 1 nM to 20 μM concentration of the components can be used, with the volume of 1 to 1000 μL. A ligase concentration of 1 to 100,000 U can be used. The temperature of ligase reaction may be 15 to 50 degrees C.

The tacrolimus DNA-nanostructures described herein can be used to detect and/or quantify tacrolimus. The DNA-nanostructure can be contacted with a sample containing or suspected to contain tacrolimus. The sample can be a fluid. Solid samples of interest can be put into a liquid mixture or solution for analysis. Samples can be obtained from any suitable source, including but not limited to, a subject or an inanimate object or source (e.g. bodily fluid, such as blood, urine, etc.). In some aspects, the sample is a complex sample (e.g. containing many types of compounds, molecules, and the like), such as blood or a component thereof (e.g. serum or plasma). In some aspects the sample can be filtered by a suitable method prior to contact with the DNA-nanostructure. Suitable filtering methods include, but are not limited to, size separation-based methods (e.g. membrane-based, chromatography, and electrophoretic methods), charge separation-based methods e.g. membrane-based, chromatography, and electrophoretic methods), affinity purification methods (e.g. antibody, aptamer, magnetic, etc. based purification methods).

After contacting the DNA-nanostructure with the sample that can contain or is suspected of containing tacrolimus, in the presence of the tacrolimus-specific binding agent (e.g., antibody, apatamer, etc.), including pre-treating the sample with the tacrolimus-specific binding agent, any unbound tacrolimus-specific binding agent may then bind to tacrolimus coupled to the DNA nanostructure, as illustrated in FIG. 1B. As shown in this example, binding of tacrolimus-specific binding agent to the tacrolimus on the tethered (e.g., to electrode) DNA nanostructures results in a change in tethered diffusion of the DNA-nanostructure, as previously described. The signal produced from the signal moiety can be measured and used to indicate presence (or absence) of tacrolimus in the sample and/or quantify an amount of tacrolimus in the sample.

The assay can include any suitable number of rinses or washes to remove unbound tacrolimus-specific binding agent between steps of sample incubation and signal measurement. In some aspects the number of washes can range from 1 to 100. Suitable buffers can include, but are not limited to, phosphate buffered saline, HEPES, cell media, Tris, Tris-EDTA, or any buffer known by those skilled in the art that will not interfere with the measurement of interest.

As previously discussed, the detection of the signal moiety can occur directly (e.g. measuring a signal produced by the moiety itself or indirectly (e.g. a signal produced by something that the signal moiety interacts with to produce a detectable signal when a stimulating interaction occurs). For direct detection, the signal molecule can produce an optical signal (e.g. florescence) that can be detected and measured by a suitable device. Suitable devices include, but are not limited to, fluorescence spectrometer, microscope, optical photometer, laser-induced fluorescence system, confocal fluorescence system, single-molecule detection apparatus, time-resolved fluorescence instrument, polarized fluorescence instrument, or any instrument capable of exciting the label and quantifying photons known to those skilled in the art. In aspects, where the signal is directly detected from the signal moiety, detection and/or quantification can be made by directly measuring a signal output from the signal moiety. A change in 3D position or frequency of a change in position over time can be measured by measuring the optical signal at a stationary position. In some aspects, an optically active signal moiety is always producing an optical signal and thus measuring the optical signal at a stationary position allows detection of binding of an analyte to the DNA-nanostructure. In some aspects, a signal is produced by an optically active signal moiety via FRET or other proximity- or resonance-based signal production method. In such aspects the signal can still be produced from the signal moiety, but only when it is in effective proximity to an energy donor. When this occurs, the signal molecule (or energy acceptor) can reach an excited state and produce an optical signal, which can be detected and measured as previously described. In some aspects, the DNA-nanostructure can be coupled to a support structure that can include a photodetector and a suitable energy donor molecule. When the signal moiety that is an energy acceptor comes in proximity to the suitable energy donor molecule, the signal moiety can produce an optical signal that can be detected by the photodetector. A change in the signal and/or frequency of signal production can be used to determine presences and/or amount of tacrolimus.

The assay can be configured such that the signal is indirectly produced from the signal moiety. In these aspects, the signal moiety causes the production of a signal by something else (e.g. electrode, energy acceptor optically active molecule etc.) by reacting with another molecule and/or electrode. In some aspects, the signal moiety is a redox molecule that produces a chemical change (e.g. a redox reaction) with a suitable surface or other molecule which can be translated into a signal by an electrode and/or detector. In some aspects, the signal moiety is an energy donor molecule that can react with an optically active energy acceptor that can be coupled to a support and/or photodetector and stimulate production of a detectable optical signal from the optically active energy acceptor via energy transfer from the signal moiety to the energy acceptor molecule when they are in effective proximity to each other. The signal produced from the energy acceptor molecule can be detected by, e.g., a photodetector. A change in the signal and/or frequency of signal production either produced via a chemical (e.g. redox) reaction or optically can be used to determine presence and/or quantify amount of an analyte of interest as previously discussed. In some aspects, a change in the electrochemical reaction rate can be used to determine the presence and/or quantify the amount of an analyte of interest. This can be measured in some aspects by a change in the current, such as an SVW current, that can be applied to an electrode.

In aspects, properties of the redox molecules can be interrogated with any electrochemical quantification technique, including but not limited to cyclic voltammetry (CV), linear sweep voltammetry, pulse voltammetry, chronoamperometry, square wave voltammetry (SWV), differential pulse voltammetry (DPV), AC voltammetry, fast-scan CV, etc. For interrogating the DNA-nanostructure, one possible algorithm to follow is to first measure the initial signal without analyte present. The introduction of tacrolimus-specific binding agent will then induce binding of the tacrolimus-specific binding agent to the tacrolimus on the DNA nanostructure, thereby shifting the output from the initial level. The percentage of shift or the magnitude of the shift, compared to the initial signal, may be proportional to the concentration of tacrolimus in the sample.

The DNA-nanostructure described herein can be more sensitive than other conventional DNA based electrochemical assays for the same analyte. This can be due in part to the structural configuration of the DNA-nanostructure. The dynamic range of the DNA-nanostructure based assay described herein can be range from the nanomolar range to the micromolar range. The dynamic range of the DNA-nanostructure based assay can range from about 1 nM to 10 μm. The dynamic range of the DNA-nanostructure based assay described herein can be from about 2 nm to 100 nm. The dynamic range of the DNA-nanostructure based assay described herein can range from about 1 μm to about 8 μm.

Components of the assay described herein can be provided as a combination kit. The combination kit can include a DNA-nanostructure as described herein and a tacrolimus-specific binding agent (e.g., a solution containing tacrolimus-specific binding agent). The kit can also include solutions, diluents, buffers, reagents, containers, membranes, plates (e.g. 6, 12, 24, 48, 64, 96, 384 well plates), that can be used in sample preparation and/or assay performance.

The methods and apparatuses described herein may relate to nucleic acid-based nanostructures linked to a tacrolimus molecule that may be used to detect and/or quantify tacrolimus in a test sample or samples. As described above, the nucleic acid-based nanostructure may be formed on the surface of a gold electrode or other substrate to create a biosensor. The nanostructure may be conjugated to tacrolimus (or in any of these variations, a small-molecule that compete with tacrolimus for binding to a tacrolimus binding partner) via any appropriate chemical method, including “click” chemistry, to setup a competition-based sensing mechanism, as described herein.

For example, the tacrolimus (or a small-molecule that can compete with tacrolimus for binding of a tacrolimus-specific binding partner) may be conjugated to the nucleic acid-based nanostructure by cycloadditions (e.g., 1,3-dipolar cycloadditions, hetero-Diels-Alder cycloadditions, etc.), Nucleophilic ring-openings (e.g., openings of strained heterocyclic electrophiles, such as aziridines, epoxides, cyclic sulfates, aziridinium ions, episulfonium ions, etc.), carbonyl chemistry of the non-aldol type-examples (e.g., formations of ureas, thioureas, hydrazones, oxime ethers, amides, aromatic heterocycles, etc.), and/or additions to carbon-carbon multiple bonds (e.g., epoxidations, aziridinations, dihydroxylations, sulfenyl halide additions, nitrosyl halide additions, and certain Michael additions). For example, the tacrolimus may be attached by a copper(I)-catalyzed azide-alkyne cycloaddition (CuAAC), a Thiol-ene reaction, Diels-Alder reaction and inverse electron demand Diels-Alder reaction, [4+1] cycloadditions between isonitriles (isocyanides) and tetrazines, nucleophilic substitution (especially to small strained rings like epoxy and aziridines), etc.

In any of the variations described herein a tacrolimus molecule or a modified version of a tacrolimus molecule may be conjugated to the nucleic acid-based nanostructure. A modified version of a tacrolimus molecule includes molecules that compete with tacrolimus for binding of a tacrolimus-specific binding partner. FIG. 7 illustrates the structure of tacrolimus that includes a tether moiety that may couple the tacrolimus to the DNA nanostructure, as described herein. FIG. 8 illustrates examples of other tacrolimus antigen molecules that may be used, and FIG. 9 shows examples of the immunophilin-binding immunosuppres sant drugs, including tacrolimus. These molecules may be specifically bound by an immunophilin-binding immunosuppressant drug binding agent (e.g., a tacrolimus-specific binding agent) that also binds to the immunophilin-binding immunosuppressant drug (e.g., tacrolimus) in the subject's blood. Any of the immunophilin-binding immunosuppressant drugs shown in FIG. 9, or metabolites or synthetic analogs of these (e.g., a tacrolimus metabolites and synthetic analog such as those shown in FIG. 8, which is a non-exhaustive list) may be use as described herein, including may be coupled to the DNA nanostructures, e.g., by including a linking or coupling moiety similar to that shown in FIG. 7 or as otherwise described herein.

In use, the binding of a tacrolimus binding partner (which may be an antibody, aptamer or several antibodies or aptamers or a combination) to the nanostructure-conjugated tacrolimus changes the signal readout from the nanostructure. This signal readout may be electrochemical, and is typically based on the chemical signaling molecule that the nanostructure is labeled with. For example, tacrolimus in the sample may compete with the nanostructure-conjugated tacrolimus antigen molecule for binding to tacrolimus-specific binding agent in the solution, resulting in a signal that is proportional to the concentration of tacrolimus in the sample, allowing for quantification of the tacrolimus in the sample.

In some variation, the assay may combine readouts from several variations of the biosensors, in which each variation may be configured to have a different binding partner concentration, incubation time or other unique reaction conditions, which may allow for a range of concentration sensitivities and/or may allow the method or apparatus to be tuned to a specific range. A combination of different variations of biosensors may be used, and the resulting signal analysis may be performed via an algorithm that allows for quantification of a wide range of tacrolimus concentrations in biological or non-biological test samples.

### EXAMPLES

FIGS. 1C-1D, 2 and 3A-3D illustrate the use of a DNA nanostructure including tacrolimus to detect tacrolimus as described above. In this example the DNA nanostructure was tethered to an electrode and raw data (including Vₛₜₑₚ and I_{diff}) was analyzed, and a moving average was applied. The maximum current was used as the peak height. Signal suppression and signal change was calculated. Equations 1 and 2, below, were used to calculate signal suppression and change, where iₚ (initial) is the peak height of the initial current measurement (before target incubation) and iₚ (final) is the peak height of the final current measurement (after target incubation).

$$Signal Suppression ( \% ) = - 100 \times \frac{{i}_{p} ( final ) - {i}_{p} ( initial )}{{i}_{p} ( initial )} \qquad ( Eq . 1 )$$

$$Signal Charge ( \% ) = 100 \times \frac{{i}_{p} ( final ) - {i}_{p} ( initial )}{{i}_{p} ( initial )} \qquad ( Eq . 2 )$$

As described above, a DNA nanostructure including tacrolimus was generated and attached at a fixed distance from a support surface and configured to electrochemically report a variety of binding interactions. Such a nanostructure undergo a change in mass upon binding, which shift the tethered diffusion, resulting in electrochemical signal change. As illustrated in FIG. 1B, this results in the competitive assay in which the amount of tacrolimus-specific binding agent (e.g., antibody, apatamer, etc.) is competed away from the tacrolimus in a sample.

In FIGS. 1C and 1D, measurements taken at either 15 min or 60 min both show that as concentrations of tacrolimus increase, the signal decreases, as there is less signal suppression at higher concentrations (in which less tacrolimus-specific binding agent is available to reduce the tethered diffusion rate (as illustrated in FIG. 1B). In FIG. 1D, the signal suppression increases with time for a particular concentration of tacrolimus (e.g., 0 nM or 50 nM).

The assays for tacrolimus described herein may be sensitive for very low concentrations of tacrolimus, including over a range that is therapeutically relevant, such as between 1-30 nM. Sensitivity may be increased or adjusted by reducing the amount of tacrolimus-specific binding agent (e.g., reducing the concentration of tacrolimus-specific binding agent). For example, a concentration of tacrolimus-specific binding agent of less than 100 nM, e.g., 50 nM or less, 30 nM or less, 20 nM or less, 15 nM or less, etc. Preliminary results show that lower concentrations of tacrolimus-specific binding agent results in more sensitivity.

In any of these assays the tacrolimus-specific binding agent may be pre-incubated in the sample for a predetermined length of time (e.g., 1 min or more, 2 min or more, 5 min or more, 10 min or more, between 1-3 min, between 1-5 min, between 2-5 min, between 5 min-10 min, etc.).

In some variations, the concentration of tacrolimus-specific binding agent may approximately match or be slightly less than the target concentration of tacrolimus in the sample. For example, for a sensitivity range of tacrolimus of between about 5 nM to 30 nM, the concentration of tacrolimus-specific binding agent in the sample may be between about 100 nM to about 15 nM (e.g., about 15 nM), below the highest anticipated concentration (target concentration). For example in some variations, to measure less than 30 nM, the tacrolimus-specific binding agent concentration may be less than 50 nM (e.g., 10 nM to 60 nM).

As shown in FIG. 1B, the timing for the assay may be selected to adjust the sensitivity. For example, in FIG. 1B, the assay performed at 15 min is more sensitive than at 60 min incubation. Longer incubation time may allow unbound tacrolimus-specific binding agent to interfere with the assay. Thus, in some variations, it may be preferred to perform the assay (e.g., the pre-incubation) for less than 30 min (e.g., less than 25 min, less than 20 min, less than 15 min, etc.).

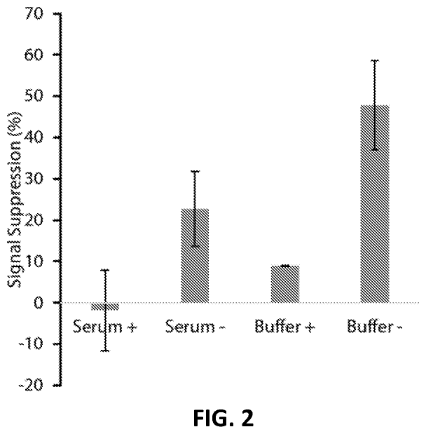

In these examples, the percent of signal suppression (using, etc., 70-75 Hz for measurement) was determined as described above. As shown in FIG. 2, the comparable effect were seen when comparing between serum (e.g., whole serum) and buffer, showing a significant reduction in the signal suppression when tacrolimus was present (+) versus when it was absent (−) in the blood serum or buffer samples. In this example, the response in serum is typically lower than buffer, the probe (e.g., the DNA nanostructure probe) is stable in serum. In FIG. 2, the serum was virtually un-diluted (e.g., between 98-99% serum).

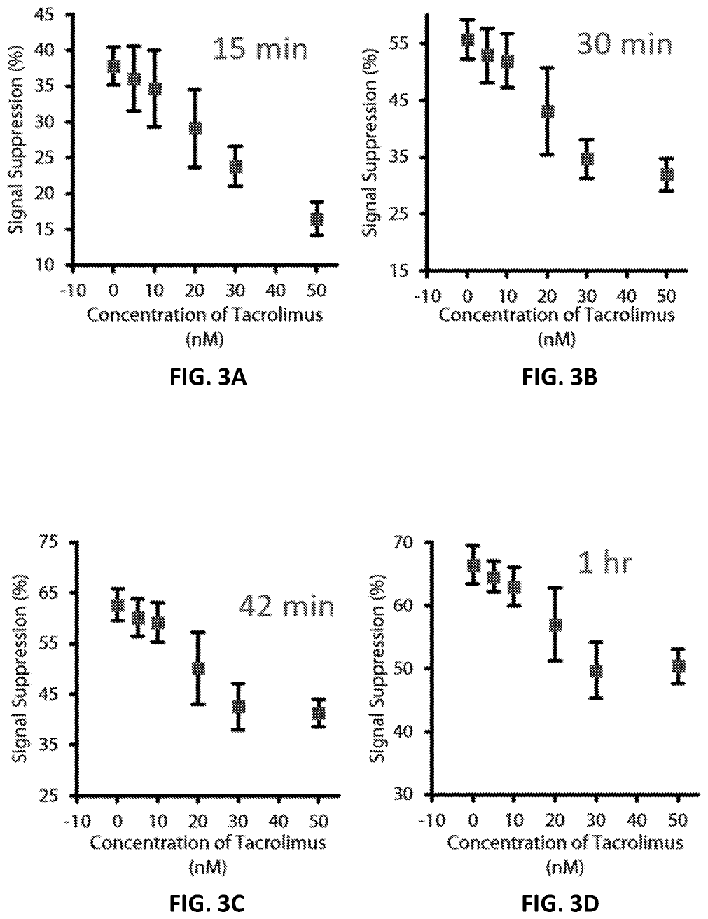

FIGS. 3A-3D illustrate signal suppression at different concentrations of tacrolimus at different times, e.g., 15 min (FIG. 3A), 30 min (FIG. 3B), 42 min (FIG. 3C) and 1 hour (FIG. 3D). These results are similar to those shown in FIG. 1C, described above.

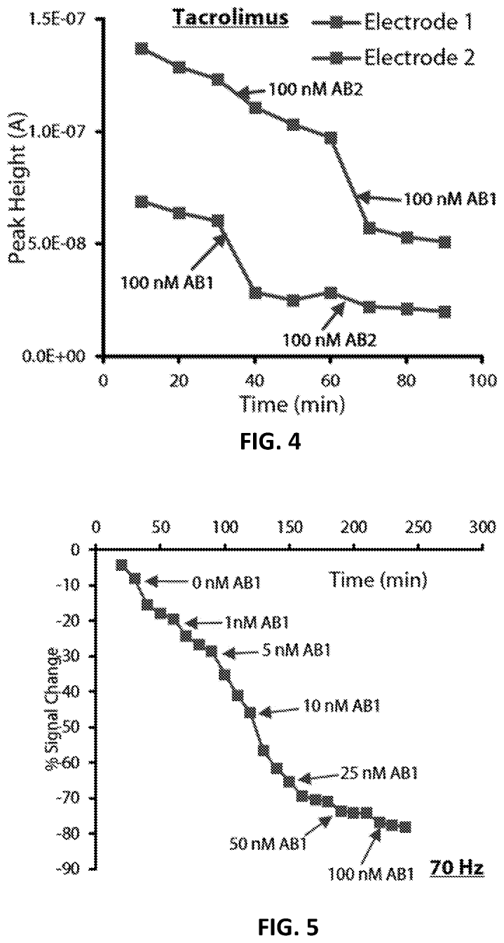

As mentioned above, any appropriate tacrolimus-specific binding agent may be used. For example, in some variations the tacrolimus-specific binding agent is an antibody directed against tacrolimus. FIG. 4 shows a comparison of the effects of different tacrolimus-specific binding agents (e.g., different antibodies, “AB1” and “AB2”). In this example, AB1 is tacrolimus 32 antibody (a polyclonal antibody raised in sheep using Tacrolimus(32)-BTG as the immunogen. AB2 is tacrolimus 22 (also a polyclonal sheep antibody, but using Tacrolimus(22)-BTG as the immunogen). In this example, although there was some variation between the different electrodes (electrode 1 and electrode 2), which may be corrected for, AB 1 more potently bound tacrolimus and showed an effect.

FIG. 8 illustrates the effect of different concentrations of tacrolimus-specific binding agent (e.g., AB1) in an assay. Although there was not a significant difference between about 50-100 nM of tacrolimus-specific binding agent, concentration rages of between about 5-25 nM (e.g., about 15 nM) showed a high sensitivity for the assay.

In all of these examples, the frequency applied to interrogate the tacrolimus DNA nanostructure was between about 15-100 Hz. 70 Hz was chosen in most cases, because it demonstrated a good percentage of suppression.

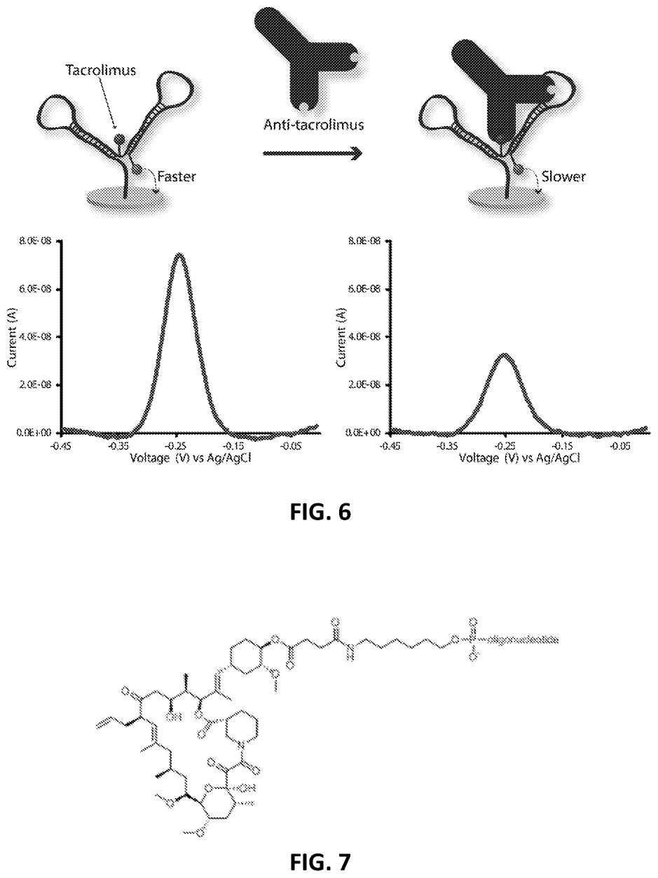

FIG. 6 shows one example of a voltometric tacrolimus-specific binding assay, showing that in the absence of tacrolimus, the current peak was between about 75-80 nA (nanoAmps). In the presence of the tacrolimus-specific binding agent, the current dropped to between 30-40 nA.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1-5. (canceled)

6. A biosensor for detecting tacrolimus, the biosensor comprising:
a polynucleotide molecule comprising:
a first hairpin structural motif, and
a second hairpin structural motif;
a tacrolimus antigen molecule conjugated to the polynucleotide molecule, wherein the tacrolimus antigen is one of: a tacrolimus molecule, a synthetic analog of tacrolimus or a tacrolimus metabolite;
a signal moiety coupled to the polynucleotide, wherein the signal moiety and the tacrolimus antigen molecule are in an effective proximity to each other so that a tethered diffusion of the signal moiety is altered when a tacrolimus-specific binding agent binds to the tacrolimus antigen molecule; and
a conductive substrate to which the polynucleotide molecule is tethered.

7. The biosensor of claim 6, wherein the signal moiety is a redox molecule.

8. The biosensor of claim 7, wherein the signal moiety is methylene blue.

9. The biosensor of claim 6, wherein the tacrolimus antigen molecule is conjugated to the polynucleotide molecule between the first hairpin structural motif and the second hairpin structural motif.

10. The biosensor of claim 6, further comprising a processor electrically coupled to the conductive substrate and configured to detect a change in the tethered diffusion of the signal moiety.

11. The biosensor of claim 6, further comprising a plurality of polynucleotide molecules each including a tacrolimus antigen molecule conjugated between a pair of hairpin structural motifs and a signal moiety conjugated to an end of each polynucleotide, wherein the plurality of polynucleotide molecules are tethered to the conductive substrate.

12. The biosensor of claim 6, further comprising a linker tethering the polynuclotide to the conductive substrate.

13. The biosensor of claim 6, wherein the polynucleotide molecule has a sequence that is 50-100% identical to any one of SEQ ID NOs: 7-8.

14. (canceled)

15. A system comprising:
a plurality of polynucleotide molecules each including a pair of hairpin structural motifs, a tacrolimus antigen molecule conjugated to each polynucleotide molecule, and a signal moiety conjugated to an end of each polynucleotide, wherein the tacrolimus antigen is one of: a tacrolimus molecule, a synthetic analog of tacrolimus or a tacrolimus metabolite;
a solution of a tacrolimus-specific binding agent;
an electrode, wherein the plurality of polynucleotide molecules are tethered to the electrode; and
a processor in electrical communication with the electrode, wherein the processor is configured to detect a change in a tethered diffusion of the signal moieties conjugated to the plurality of polynucleotides.

16. The system of claim 15, wherein the signal moieties conjugated to the plurality of polynucleotides are redox molecules.

17. The system of claim 16, wherein the signal moieties are each methylene blue.

18. (canceled)

19. The system of claim 15, wherein the tacrolimus-specific binding agent comprises an antibody specific to tacrolimus.

20. The system of claim 15, wherein the tacrolimus-specific binding agent comprises an aptamer specific to tacrolimus.

21. The system of claim 15, wherein the polynucleotide molecule has a sequence that is 50-100% identical to any one of SEQ ID NOs: 7-8.

22. (canceled)

23. A method of identifying tacrolimus in a sample, the method comprising:
exposing a sample mixture including the sample to a biosensor comprising a signal moiety and a tacrolimus antigen molecule coupled to a polynucleotide molecule, wherein the tacrolimus antigen molecule is one of: a tacrolimus molecule, a synthetic analog of tacrolimus or a tacrolimus metabolite,
wherein either the sample or the biosensor is pre-incubated with a tacrolimus-specific binding agent prior to exposing the sample mixture to the biosensor;
detecting a change in a tethered diffusion of the signal moiety relative to a surface; and
outputting the presence and/or level of tacrolimus in the sample based on change in tethered diffusion of the signal moiety.

24. (canceled)

25. The method of claim 23, wherein exposing the sample mixture to the biosensor comprises exposing the sample mixture to the biosensor, wherein the biosensor comprises a plurality of polynucleotide molecules each including tacrolimus antigen molecule conjugated to each polynucleotide molecule and the signal moiety conjugated to an end of each polynucleotide molecule.

26. The method of claim 23, wherein the tacrolimus-specific binding agent in the sample mixture is at a concentration of 200 nM or less.

27. The method of claim 23, wherein the tacrolimus-specific binding agent in the sample mixture is at a concentration of between 1 to 100 nM in the sample mixture.

28. (canceled)

29. The method of claim 23, wherein detecting a change in a tethered diffusion of the signal moiety relative to a surface comprises detecting a change in an electrochemical output of the biosensor, wherein the biosensor comprises an electrode to which the polynucleotide molecule is tethered.

30. The method of claim 23, wherein the biosensor comprises a plurality of polynucleotide molecules each including tacrolimus conjugated between a pair of hairpin structural motifs and the signal moiety conjugated to an end of each polynucleotide, wherein the plurality of polynucleotide molecules are tethered to an electrode.

31-39. (canceled)
